(19) United States
(12) Patent Application Publication
(10) Pub. No.: US 2014/0199698 A1
(43) Pub. Date: Jul. 17, 2014

(54) METHODS OF PREDICTING AND DETERMINING MUTATED mRNA SPLICE ISOFORMS

(71) Applicants: Peter Keith Rogan, London (CA); Eliseos John Mucaki, London (CA)

(72) Inventors: Peter Keith Rogan, London (CA); Eliseos John Mucaki, London (CA)

(21) Appl. No.: 14/154,905

(22) Filed: Jan. 14, 2014

Related U.S. Application Data
(60) Provisional application No. 61/751,975, filed on Jan. 14, 2013.

(51) Int. Cl. C12Q 1/68

(52) U.S. Cl. CPC C12Q 1/68
USPC 435/6.11

(57) ABSTRACT

Mutations that affect mRNA splicing often produce multiple mRNA isoforms containing different exon structures. Definition of an exon and its inclusion in mature mRNA relies on joint recognition of both acceptor and donor splice sites. The instant methodology predicts cryptic and exon skipping isoforms in mRNA produced by splicing mutations from the combined information contents and the distribution of the splice sites and other regulatory binding sites defining these exons. In its simplest form, the total information content of an exon, R_{i,total}, is the sum of the information contents of its corresponding acceptor and donor splice sites, adjusted for the self-information of the exon length. Differences between R_{i,total} values of mutant versus normal exons are consistent with the relative abundance of these exons in distinct processed mRNAs. Predictions of splicing mutations based on R_{i,total} are highly concordant with published expression data demonstrating alterations in the structures and relative abundance of the mRNA transcripts derived from these mutations.

### RELATED APPLICATIONS

This application claims priority of U.S. Provisional Application No. 61/751,975 filed on Jan. 14, 2013, the content of which is hereby incorporated into this application by reference.

### BACKGROUND OF THE INVENTION

I. Field of the Invention

The present method relates to methods for assessing changes in expression level of a gene and to in silico prediction of cryptic and exon skipping isoforms in mRNA produced by splicing mutations by combined information contents and distribution of the splice sites defining these exons (exon definition analysis). The method allows for streamlining assessment of abnormal and normal splice isoforms resulting from such mutations.

II. Description of the Related Art

mRNA processing mutations, which are responsible for a wide range of human diseases (Divina et al., 2009), alter the abundance and/or structures of mature transcripts. These mutations often occur proximate to exon/intron boundaries, but are frequently found at other sequence locations within introns or exons. Mutations which abolish or weaken recognition of natural splice acceptor or donor sites often produce transcripts lacking corresponding exons or activate adjacent cryptic splice sites of the same phase. Alternatively, mutations activate cryptic splice sites whose strength exceeds existing natural sites elsewhere in the unspliced transcript. The resultant molecular phenotypes may include isoforms with altered exon length and, in some instances, reduced or leaky expression of normal isoforms. We propose an approach based on information theory to predict the structures and approximate abundance of the output molecules generated directly or indirectly by splicing mutations.

Berget's exon definition model (Berget, 1995) provides a mechanism for recognizing multiple small exons against a background of considerably larger intronic sequences. Accurate exon recognition can be complicated by pseudo-exonic structures present in introns that mimic natural exon structures (Ibrahim et al., 2005). To discriminate between these structures, accurate spliceosomal recognition relies on relatively high affinities of the recognition sequences in natural exons and the presence of other splicing regulatory elements. Exons and adjacent introns also contain splicing enhancer (ESE, ISE) and silencer (ESS, ISS) sequences close to or overlapping constitutive splice sites, which may assist or suppress exon recognition through interactions with additional proteins (Berget, 1995; Graveley and Maniatis, 1998). Recognition of an exon may therefore depend to some degree on the combined effects of each of these proteins (Goren et al., 2010), however the factors that recognize the acceptor and donor splice sites are often sufficient (Hwang and Cohen, 1997).

Information theory can be used to measure the conservation of nucleotide sequences bound by individual proteins or protein complexes. In splicing, information theory-based models of donor and acceptor splice sites reveal which nucleotides are permissible at both highly conserved and variable positions in individual sites (Schneider, 1997; Robberson et al., 1990; U.S. Pat. No. 5,867,402). These sequences are recognized prior to intron excision, these recognition events are concerted, and related to the binding strength of the spliceosome-splice site interaction (Berget, 1995). The strengths of spliceosome-splice site interactions are related to the corresponding individual information content, Rᵢ, of the RNA sequence (Rogan et al., 1998). As disclosed here, an exon may be defined by the cumulative Rᵢ values of each of these distinct binding sites contributing to exon recognition (R_{i,total}), based on the fact that information is additive for independent sources of uncertainty (Jaynes 1957).

Previously described bioinformatic methods that predict the effects of mutations that could alter mRNA splicing generally examine the effect of a single gene variant in situ, at or proximate to the mutation itself. Among these programs are Cryp-SKIP (http://cryp-skip.img.cas.cz/), SpliceScan II (Churbanov et al. 2010), Annovar pipeline, Bayesian sensor (Churbanov et al. 2006) and SpliceScan tool (Churbanov et al. 2006), Alamut software (http://www.interactive-biosoftware.com/alamut.html) that includes (SSF, Max-EntScan, NNSPlice, and GeneSplicer). Alamut software has been used in a recent study of aberrant splicing prediction (Thomassen et al. 2012) and has been found to be sensitive, but not specific (Spurdle et al. 2012). None of these computations make reference to, incorporate, or anticipate exon recognition processes. While machine learning methods have been developed to predict alternatively spliced transcripts, a natural process that occurs in cells with a normal genotype (Barash et al, 2010), these ad hoc methods are not supported by a rigorous theoretical framework that relates the predicted isoforms to thermodynamic binding affinity and thus cannot be used to analysis of the relative abundance of different isoforms.

CRYP-SKIP is another bioinformatic method which employs multiple logistic regression to predict the two aberrant transcripts from the primary sequence (Divina et al., 2009). It predicts the overall probability of cryptic splice-site activation as opposed to exon skipping, which has some resemblance to exon definition. However, the online resource developed for this method (http://cryp-skip.img.cas.cz/) does not take into consideration the impact of mutations. Although a user can simply analyze the wildtype and mutated sequences individually and compare them manually, such method is not based on information theory, nor does it use the gap surprisal function to factor exon size penalties.

Fairbrother described a method for predicting the effects of mutations on splicing. US Patent application Publication No. US2013/0096838 A1. However, Fairbrother fell short of teaching how to determine the relative level of each spliced isoform as a result of the mutation(s). Moreover, Fairbrother did not consider the contribution of splicing regulatory sequence to the relative abundance of RNA splice isoforms.

### SUMMARY

The present disclosure provides methods for assessing changes in expression level of a gene due to mutation(s) that may affect mRNA splicing. This disclosure also provides methods for predicting cryptic and exon skipping isoforms in mRNA produced by splicing mutations by combined information contents and distribution of the splice sites defining these exons (exon definition analysis).

In contrast with splice sites across an intron, cognate pairs of donor and acceptor splice sites from the same exon tend to be separated by a narrow range of distances in the unspliced transcript. Single exon recognition tends to be constrained by preferred distances between the U2 and U1 spliceosomal binding sites across the same exon (Hwang and Cohen, 1997). A model to define exon sequences that incorporates the information contents of both splice sites and preferences for certain exon lengths of all natural exons has been previously presented (Rogan, 2009). A general approach is used that minimized entropy of a pair of binding sites separated by a variable length interstitial sequence. Given a set of exons flanked on either side by 100 nucleotides (nt) intron sequences, the most accurate model (99% correctly detected exon boundaries) was derived by bootstrapping sets of 4000 sequences with left (acceptor) and right (donor) sites of 31 (9.7 bits) and 15 nts (8.1 bits) in length. Efforts are used to ensure that pairs of splice sites of opposite polarity are derived from the same exon by incorporating the surprisal function (Tribus, 1961), also termed self-information by Shannon (Cover and Thomas, 2006), which corrects for both frequent and uncommon or rare inter-site distances that are unlikely to form an exon. This is based on the observation that long internal exons are recognized inefficiently (Robberson et al., 1990), though they do occur (1115 known internal exons >1000 nt; (Bolisetty and Beemon, 2012). The total exon information content (R_{i,total}) is significantly reduced by this gap surprisal value, if either the predicted exon length is suboptimal or splice site pairs are derived from different exons, but is nearly unchanged for common exon lengths.

The present disclosure provides a novel method for determining and predicting the effect of a splicing mutation on the relative abundance of natural and cryptic splice isoforms using the exon definition model. The method may contain, among others, the following steps:

(a) Calculating the information content of all donors and acceptors within a given region, before and after mutation;
(b) Pair all donors to all acceptors predicted in (i) and apply a gap surprisal term that depends on the transcriptome-wide distribution of the lengths separating them;
(c) Calculate the total information content of every potential exon before and after mutation, and ranking them in descending order post-mutation; and
(d) Categorize each predicted exon based on their use of naturally used donor and acceptor splice sites using an database containing publicly-available GenBank and RefSeq cDNA accessions.

In one embodiment, all methods disclosed herein may include a step of extracting mRNAs or proteins from at least one cell expressing the gene to determine the most abundant mRNA splice isoform of the gene, thus allowing the assessing of changes in expression level of the gene. In one aspect, the extracting step may be performed by extracting mRNAs from said at least one cell and by determining the sequence of one or more mRNA molecules derived from the gene. In another aspect, the extracting step is performed by extracting proteins from said at least one cell expressing said gene and by determining the sequence of one or more protein molecules derived from the gene of interest.

In another embodiment, all methods may include a step of introducing the gene into at least one cell and extracting mRNAs or proteins from the at least one cell expressing the gene to determine the most abundant mRNA splice isoform of the gene, thus allowing the assessing of changes in expression level of the gene.

In another embodiment, the steps (a)-(d) described above may be preceded by a step of generating a genomic polynucleotide sequence of the gene of interest. In one aspect, the genomic polynucleotide sequence may be generated by isolating genomic DNA from a cell containing the gene and by sequencing the isolated genomic DNA using PCR, conventional sequencing or other sequencing techniques, such as mass spectrometry.

It is an object of the present disclosure to use information-theory based exon definition models to generate testable predictions of splice isoforms activated and deactivated by splicing mutations, which can reveal splice isoforms that have not been previously described.

It is an object of the present disclosure to be able to predict relative abundance of these wild-type and mutated splice forms comparison of total exon information values.

It is an object of the present disclosure to factor splicing mutation-directed changes in splicing enhancers and silencers (small nuclear ribonucleoproteins; snRNPs) into the total exon information calculation. A second snRNP-specific gap surprisal function, which is based on the common distance between a natural splice site and the nearest predicted splicing enhancer of the same type, would also be applied.

It is disclosed here a novel approach to predict the molecular phenotype of a splicing mutation, producing a probable set of splicing isoforms expressed in mutation carriers. The system is based on information theory-based methods that accurately quantify binding site affinity (Schneider, 1997; Rogan et al., 1998). Non-expressed or very low expression exons are filtered out by correcting for suboptimal exon lengths and eliminating incorrectly ordered splice sites.

It is also shown here a simple model for exon definition based on constitutive splice sites, although the theory for extensible framework for incorporation of multiple splice site recognition sequences is derived. Exon definition-based predictions were compared to known splicing mutations with published mRNA studies, and these predictions were found to be highly concordant (FIG. 8). These mutations were sourced from our previous publications so that information theory based modelling of individual splice sites could be compared with exon definition (Rogan et al., 1998; Mucaki et al., 2011).

Information analysis correctly predicted several types of splicing abnormalities in different genes. There were 31 mutations which resulted in formation of one or more cryptic exons (FIG. 8). Exons using these cryptic splice sites were predicted for 28 of the 31 mutations, 20 of which had the highest R_{i,total} values. The other 8 mutations were ranked these cryptic splicing isoforms among the highest 6 in abundance, save one (FIG. 8 #10). Complete intron retention was reported for one mutation (#40), while 9 mutations were found to result in exon skipping only (#1, 7, 8, 11, 14, 23, 26, 37 and 41). Previously, we have shown that large changes in ΔRᵢ can result in exon skipping as well as leaky splicing (Rogan et al., 1998). All of these mutations decreased R_{i,total} of the natural exon, although in one case, the extent was marginally below significance (#14; 0.8 bits). Exon skipping was reported for mutations #7, 8, 23 and 24 rather than reduced levels of exon inclusion suggested by the exon definition analysis. These mutations reduced the predicted exon abundance by 9 to 23 fold relative to the normally spliced product. This level of expression is close to the detection limit of a minor cryptic splice isoform for most analytic methods (Rogan et al., 1998), and may explain why only exon skipping was documented for these mutations (Macias-Vidal et al., 2009; Tompson et al., 2007; Claes et al., 2002; Claes et al., 2003). Additionally, the discrepancy could simply be due to the limitations of the in vitro analyses used.

Exon definition analysis of the remaining mutations showed partial discordance to published mRNA evidence. In 3 cases, the reported cryptic site used had an Rᵢ<0 bits (#10, 15, 32). Mutation #27, R_{i,total} of the natural and the proven activated cryptic site does not quite reach the threshold for a functional site defined by information theory. In the final case (#22), the creation of a cryptic donor is predicted (2.7 bits), but the resultant 425 nt exon is not observed (R_{i,total}<0).

The development of exon definition-based mutation analysis was motivated by the desire to generate predictions that could be directly compared with laboratory expression data. In some instances, these predictions have included strong cryptic exons that have not been previously detected, possibly because the laboratory studies did not directly anticipate the corresponding splice isoforms. The level of concordance we report for previously validated splicing mutations justifies a prospective study of natural and mutant isoforms predicted by the server, in which all predicted cryptic splice isoforms are tested, and if possible, quantified. It should be feasible to implement transformative calculations to automate design of isoform specific sequence primers for quantitative expression analysis. This feature will close the circle between bioinformatic methods that predict potential splicing mutations in large scale genomic DNA sequence studies and validation with mRNA obtained from the same individuals.

In one embodiment, a method is disclosed for assessing changes in expression level of a gene of interest. In one aspect, the gene has an mRNA splice-altering mutation. In another aspect, the mutation is located within a sequence window circumscribing an exon and one or more intronic sequences of the gene, where the one or more intronic sequences are adjacent to the exon.

In another embodiment, the mutation may occur at a cryptic splice site. For instance, the mutation may be a leaky or partial splicing mutation, which causes a mutant isoform to exceed the abundance of the normal mRNA splice isoform by at least 1 bit or 2 fold. In one aspect, the mutation may result from a paucimorphic allele or an effectively null allele in which a mutant isoform exceeds the abundance of the normal mRNA splice isoform by at least 5 bit or 32 fold.

In another embodiment, the mutation may occur at a natural splice site. For example, the mutation may be a leaky or partial splicing mutation, which causes the R_{i,total} of the mutant isoform to be less than the R_{i,total} value of the normal mRNA splice isoform by at least 1 bit or 2 fold. In one aspect, the mutation may result from a paucimorphic or an effectively null allele in which the R_{i,total} of the mutant isoform is less than the R_{i,total} value of the normal mRNA splice by at least 5 bits or 32 fold.

The method may include at least the following steps (a)-(d): (a) computing and identifying changes in the individual information contents of potential donor and acceptor splice sites at each nucleotide position by computing product of the information theory-based position weight matrices and a unitary position matrix of each sequence; (b) defining potential exons by selecting every pair combination of acceptor and donor splice sites in the sequence window, and determining the gap surprisal value based on distance in nucleotides between sites comprising a pair combination, wherein, the gap surprisal value is calculated for each potential exon length based on frequency of said length in the genome as the inverse log₂ of said frequency; (c) computing the total information content, R_{i,total}, of a potential exon as the sum of the corresponding individual information contents of the acceptor and donor pair, corrected by adding the gap surprisal of an exon whose length is the distance between the donor and acceptor pair; and (d) comparing the R_{i,total} values of all potential mRNA splice isoforms of the wild-type gene and the same values after the wild-type gene sequence is mutated to determine whether the mutation alters the abundance of the mRNA isoforms containing the exon, wherein the splice isoform with the largest R_{i,total} value is predicted to be the most abundant splice isoform, and the splice isoform with the smallest R_{i,total} value is the least abundant isoform.

In one embodiment, the steps (a)-(d) described in the previous paragraph may be preceded by a step of generating a genomic polynucleotide sequence of the gene of interest. In one aspect, the genomic polynucleotide sequence may be generated by isolating genomic DNA from a cell containing the gene and by sequencing the isolated genomic DNA using PCR, conventional sequencing or other sequencing techniques, such as mass spectrometry.

In another embodiment, the comparison step (d) above may be performed by determining the relative abundance of a pair of splice isoforms by computing 2 to the power of the difference between the R_{i,total} values of each isoform.

In one aspect, the disclosed method may be specific for first exons, using a first exon-specific gap surprisal function. In another aspect, the disclosed method may be specific for last exons, using a last exon-specific gap surprisal function.

In another embodiment, the method adds a component that takes into account one or more splicing enhancer or silencer sequence elements recognized by RNA binding proteins or small nuclear ribonucleoproteins, wherein strength of at least one of the splicing enhancer or silencer sequence elements is altered due to the mutation.

In another embodiment, the method may further include a step of correcting the R_{i,total} from step (c) by taking into account one or more splicing enhancer and/or one or more silencer sequence elements recognized by an RNA binding protein or a small nuclear ribonucleoprotein, wherein strength of at least one of said splicing enhancer and/or said one or more silencer sequence elements is altered due to the mutation of the gene.

In another embodiment, a secondary gap surprisal may be applied to take into account distances between the natural splice site and each of the altered splicing enhancer and/or silencer sequence elements. In one aspect, when one or more weak binding sites overlap with a stronger binding site, proteins capable of binding to the weak sites may be essentially displaced by the protein with the higher affinity site. The weak sites may not be taken into account when applying the secondary gap surprisal.

In another embodiment, the disclosed method may also take into consideration the effects on exon definition by the mutation at binding sites for an RNA binding protein. This consideration may be accomplished by correcting the total information content (R_{i,total}) by changes in strengths of the binding sites and by applying a gap surprisal term to the computation, wherein the gap surprisal may be determined by scanning the genome for binding sites of said binding protein with a position weight matrices (PWM) to determine the frequency of each interval length between known natural sites and the nearest binding site for said RNA binding protein, separately for exons and introns. In one aspect, the PWM may be generated using known CLIP-seq libraries for said RNA binding protein generated by using chemical crosslinking methods.

### BRIEF DESCRIPTION OF THE DRAWINGS

FIG. 1 shows distribution of the R_{i,total} annotated exons. Distribution of the R_{i,total} of Annotated Exons. Histogram of R_{i,total} values for exons in the RefSeq database are illustrated for first (a), last (b), and internal exons (c).

FIG. 2 shows server input and results for BRCA1 mutation, chr17:g.412090680>A. A) User input. The window size of 200 nt increases the number of potential cryptic isoforms reported beyond the default length; B) Resulting table after applying splicing mechanism and exon abundance filters (isoforms 5-14 are not presented due to space limitations).

FIG. 3 shows structure and relative abundance of predicted isoforms. Panels: (A) The scale above shows the genome coordinates of each of the isoforms. All prospective isoforms (sorted by R_{i,total}) are scaled according to their genomic coordinates (above glyphs). The exon skipping splice form is displayed for mutations where resulting R_{i,total}<0 bits; (B and C) Plots indicating predicted pairwise (x,y axes) relative minimum fold differences in abundance (z axis) of each isoform both before and after changes in R_{i,total} due to the mutation.

FIG. 4 shows architecture of the ASSEDA server.

FIG. 5 shows flow chart of the ASSEDA server. The program flow chart of the server, with brief descriptions of the programs listed.

FIG. 6 shows Gap Surprisal distributions for constitutive splice sites of all human exons. The gap surprisals are separated by category of exon: internal (panel A), first (panel C) and last (panel D) exons of genes. To illustrate the apparent triplet periodicity of the gap surprisal function associated with open reading frames in exons of common length (50-150 nt), panel B is included.

FIG. 7 shows Gap Surprisal distributions for SF2/ASF (SRSF1) and SC35 (SRSF2) sites adjacent to constitutive splice sites in introns and exons. The gap surprisal distributions are displayed for SF2/ASF exonic (A); SF2/ASF intronic (B); SC35 exonic (C); SC35 intronic (D).

FIG. 8 shows analysis of published mRNA splice-altering mutations by information theory-based exon definition analysis.

FIG. 9 shows analysis of published regulatory ESE/ISS mutations altering mRNA splicing by exon definition analysis.

FIG. 10 shows analysis of normally spliced large (>1000 nt) exons.

FIG. 11 shows sequence logo and weight matrix of splicing regulatory sequence binding sites.

FIG. 12 shows validation of information theory based exon definition analysis-of mRNA splice-altering mutations by qRT-PCR.

FIG. 13 shows the gap surprisal distributions for ELAVL1, PTB, TIA1 and hnRNPH.

FIG. 14 shows hnRNP A1 binding site and description of information theory-based model. Panel (A) The opal codon in FANCM contained the core sequence of the novel hnRNP A1 site (Sequence logo, positions 1-3). (B) The gap surprisal function for hnRNP A1 binding sites shows that sites within exons become significantly less frequent as their distance from the natural splice site increases. (C) Sequence walkers depicting the creation of a novel 4.6 bit hnRNP A1 binding motif spanning positions 45667919-45667925.

### DETAILED DESCRIPTION

### Exon Information Content

The information content of a spliced exon may be derived from the cumulative contributions of the nucleic acid binding sites recognized by the spliceosomal machinery and the distribution distances separating binding sites within the same exon. Given a set S of n different binding sites in an exon, each of which are recognized by m different proteins, then S={xₙ, where 1≦n≦m}. The total information content, Iₛ, of all sites in S is

$${I}_{s} = {\sum }_{n = 1}^{m} {R}_{i} ( {x}_{n} ) … … … bits \qquad ( 1 )$$

The information content of each site, Rᵢ(xₙ) (measured in bits) is derived from a weight matrix (R_{iw}) representing the sequence conservation of each nucleotide in that sequence. The derivation has been presented previously (Schneider, 1997; Rogan et al., 1998).

The information contents of each set of binding sites are modified to account for the probability that these sites occur within the same exon. This requires a gap surprisal term that depends on the transcriptome-wide distribution of the lengths separating them. The gap surprisal is applied to a set of sites within the same exon. Each combination of different binding proteins (x₁, x₂ . . . ) is described by a distinct distribution. The number of different, unordered pairs of binding sites, given n different sites, correspond to (₂ⁿ), different gap surprisal terms. The gap surprisal for two binding sites (xₚ and x_{q}), separated by L nucleotides g(L_{pq}), is

g(L_{pq})=−log₂(P(L_{pq}))bits (2)

where L_{pq} is the distance between xₚ and x_{q} sites. We calculate P(L_{pq}) from experimentally validated inter site distances from human genes. Equation (4) signifies that the greater the distance between two sites, the larger the gap surprisal (greater penalty) will be, resulting in a biological reduction of larger than consensus exon length occurrence.

Denoting G(Lₛ), the total gap surprisal of (₂ⁿ) different pairs of sites in set S,

$$G ( {L}_{s} ) = \text{?} \text{?} g ( {L}_{pq} ) … … … \\ \text{?} \text{indicates text missing or illegible when filed} \qquad ( 3 )$$

The total information content (R_{i,total}) defined by combining Equations (1) and (3),

$${R}_{i , total} = {\sum }_{n = 1}^{m} {R}_{i} ( {x}_{n} ) + \text{?} \text{?} g ( {L}_{pq} ) … … … \\ \text{?} \text{indicates text missing or illegible when filed} \qquad ( 4 )$$

To calculate the R_{i,total} of an internal exon, we consider the simplest case with a constitutive set of donor and acceptor splice sites (n=2). We define x₁ as the acceptor and x₂ to be the donor site. xₙ has been extended to incorporate other types of binding sites, including splicing regulatory factors, SF2/ASF (SRSF1) and SC35 (SRSF2), that modify exon recognition. These factors act to enhance splicing when the recognition sites are located within exons (ESE) and repress splicing (ISS) if occurring in the intron adjacent to constitutive splice sites (Lim et al., 2011). The sign of this term in R_{i,total} is positive if the binding site is exonic and negative if it is intronic. The pairwise distribution of functional binding sites in the transcriptome is required to determine g(L_{pq}). For the first and last exons of a gene, R_{i,total} is the sum of the Rᵢ value of the single splice site in that exon adjusted for g(L), where L is exon length, and is based on length distributions for the corresponding terminal exons. The sign of the g(L_{pq}) term is negative for exonic locations (ESE) and reversed for intronic sites (ISS). We calculate and compare R_{i,total} values for the strengths of the constitutive splice sites in an exon prior to and after a mutation (detailed below). Isoforms with either different donor or acceptor sites may be predicted for each mutation. Because the lengths of these isoforms may vary considerably from each another, analysis of compound mutations at different gene locations has been disabled in molecular phenotypic analysis. The exon definition transformation requires at least one natural site from an exon to be contained in the predicted isoforms; thus, cryptic or pseudo-exons activated by intronic mutations are not reported. Nevertheless, the point mutation analysis capability of the ASSA server may detect these sites.

Gap Surprisal is the penalty given as per length of the exon. To correctly define the gap surprisal for a combination of splice sites, a table was constructed which relates the gap surprisal to the length of the exon. The whole genome was scanned and the frequencies of different lengths of exons occurring in the genome and their respective probability of occurrence were calculated.

According to Tribus (1961), the amount of self-information contained in a probabilistic event depends only on the probability of that event: the smaller its probability, the larger the self-information associated with receiving the information that the event indeed occurred. The self-information or surprisal I(ωₙ) associated with outcome ωₙ with probability P(ωₙ) is:

I(ωₙ)=log(1/P(ωₙ))=−log(P(ωₙ))

Here, the base of the logarithm is not specified: if using base 2, the unit of I(ωₙ) is in bits. The above definition is used to deduce gap surprisal function. The self-information or gap surprisal, g(Lₙ), of observing a pair donor and acceptor site separated by L nucleotides is −log 2(P(Lₙ)) bits. The self-information or gap surprisal, g(Lₙ), of observing a pair donor and acceptor site separated by L nucleotides is −log₂(P(Lₙ)) bits. The gap surprisal is defined as follows

Gap Surprisal=Log₂(1/probability of occurrence the exon length).

This function signifies that the greater the distance between the donor and acceptor sites, the larger the gap surprisal (greater penalty) will be, resulting in a biological reduction of larger than consensus exon length occurrence. The gap Surprisal values for different exon lengths were calculated using the above formula.

The most frequent length was assigned a gap surprisal of zero, based on the fact that splice sites separated by this distance have a highest likelihood of forming an exon. This length was 96 nucleotides (1901 occurrences among total 172250 occurrences). The frequency for this particular length 96 was: 1901/172250=0.011036. The gap surprisal for the most common, ie. preferred, constitutive exon length is 6.59 bits. To normalize all other gap surprisal terms for all other exon lengths to this value and eliminate the gap surprisal penalty for exons of 96 nucleotides, all of the penalties for all exon lengths were corrected by subtracting 6.59 bits from their respective gap surprisal values.

Total information content of either the acceptor or donor or both was found to be less than zero bits (most of these represent initial and terminal exons, as expected, since these do not contain both donor and acceptor splice sites). To successfully recognize the initial and terminal exons, a separate exon definition distribution was defined for these.

### Gap Surprisals of First and Last Exons

As the exon definition hypothesis cannot be applied for first exon since no acceptor site is defined; and for last exon since no donor site is defined, different gap surprisals were defined for selection of these exons. Separate gap surprisal tables were constructed for these exons by scanning refseq and identifying the frequencies of different lengths of first and last exons. It was observed that most frequent length of the first exon was 46 nucleotides and that of last exon was 24 nucleotides. Hence the minimum gap surprisal (0 bits) was assigned to length of 158 for the first exon and a length of 232 for the last exon.

Populating the annotation database

The ASSEDA server is based on human genome reference sequence hg19 (GRCh37), GenBank and RefSeq cDNA accessions (downloaded from genome.ucsc.edu, July 2011), and SNP (dbSNP 135) tables. Genome-wide information weight matrices for automatically curated acceptor (n=108,079) and donor (n=111,772) splice sites (acceptor_genome and donor_genome, respectively; described in (Rogan et al., 2003)), were used in the R_{i,total} calculation. The reference sequence was scanned with these matrices to determine the R_{i,total} of known natural splice sites and used to populate a MySQL database table (ALL_RI, modified from the all_mRNA.txt and the refSeqAli.txt from the UCSC genome browser).

The frequencies of different exon lengths occurring in the RefSeq database were determined for the gap surprisal calculation. Gap surprisals were normalized, based on highest frequency distance separating splice sites of opposite polarity, which was assigned G(L_{q})=0 bits. Separate distributions were compiled, respectively, for first, internal, and last exons, and stored in separate database tables. The start and end positions of first and last exons were relaxed to include any coordinate within a 200 nt window once in order to avoid duplication of exons in the gap surprisal calculation (this accounts for variation in the methods used to generate the cDNAs that are mapped onto the genomic sequence).

Incorporating Models of Splicing Regulatory Sequences into R_{i,total}

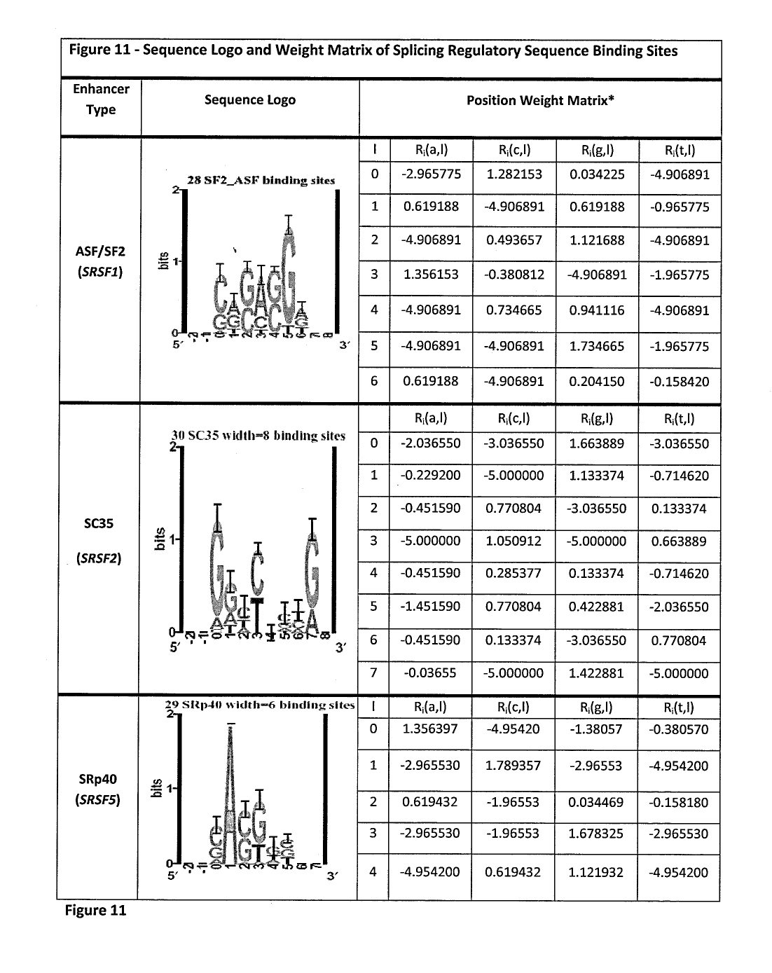

The impact of mutations in ISS or ESE's at SF2/ASF or SC35 binding sites on constitutive splicing can be predicted by selecting the option to incorporate this term into the R_{i,total} computation (on the Advanced Options page). Information weight matrices, Rᵢ(b,l), for SF2/ASF, SC35, SRp40 (SRSF5), and SRp55 (SRSF6) were derived from previously published data (Liu et al., 1998; Liu et al., 2000; Smith et al., 2006), and supplemented by experimentally-validated binding sites curated from subsequent publications (sequence logos and weight matrices are available in FIG. 11). After scanning the reference genome and locating all predicted binding sites with the SF2/ASF and SC35 Rᵢ(b,l) matrices, their distributions, g(L_{pq}) were determined separately for intronic and exonic binding sites in closest proximity to adjacent constitutive splice sites. In computing R_{i,total}, the strongest pre-existing splicing regulatory site affected by the mutation (with the highest initial Rᵢ value) is selected by the server, unless the final Rᵢ value of a second site surpasses that of the pre-existing site upon introduction of the mutation (then the second site is reported). The gap surprisal table that is applied is based on which splicing regulatory protein is selected, and the location of the site.

### Description of Server

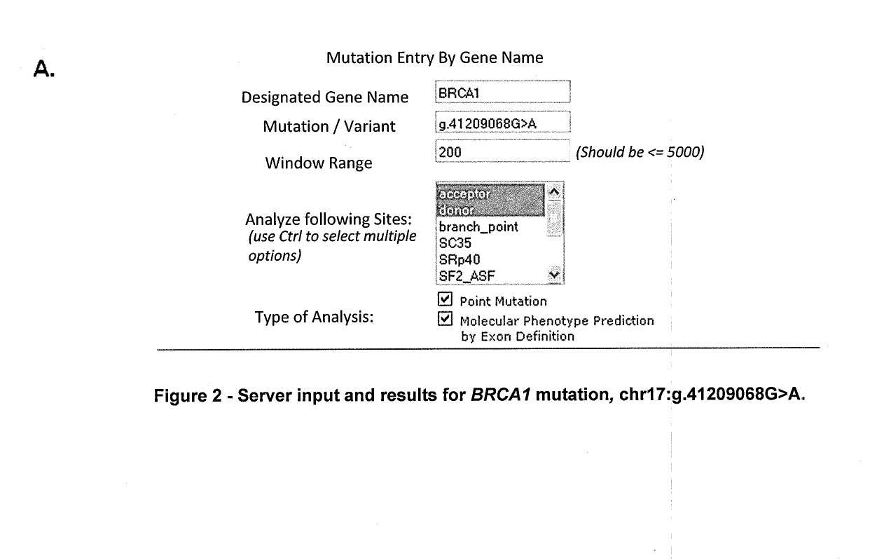

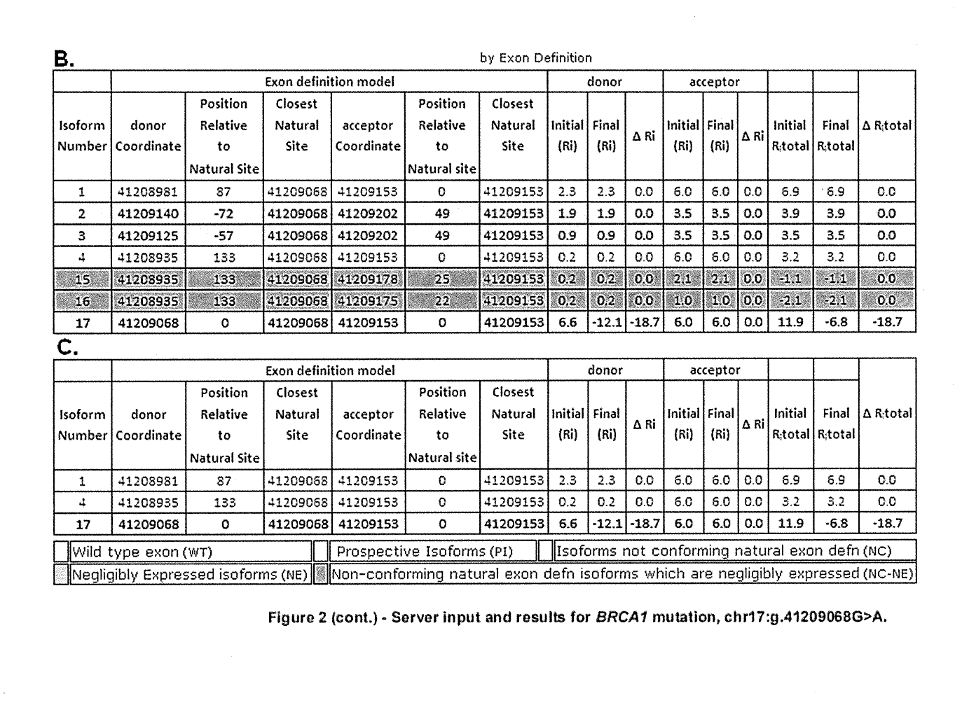

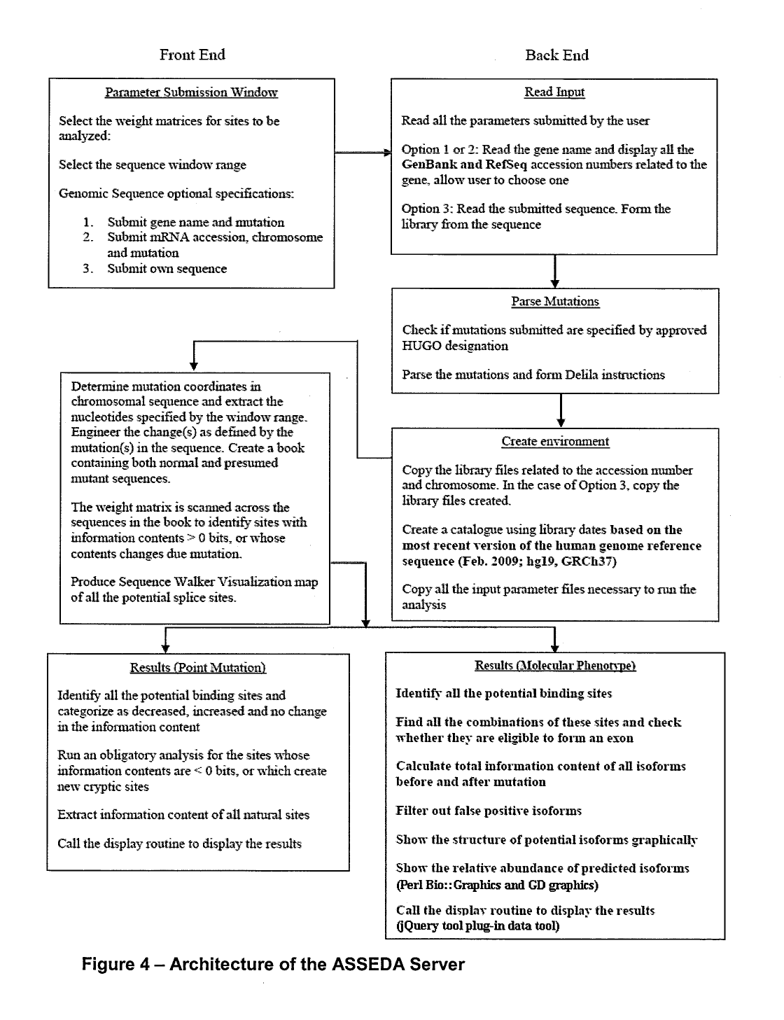

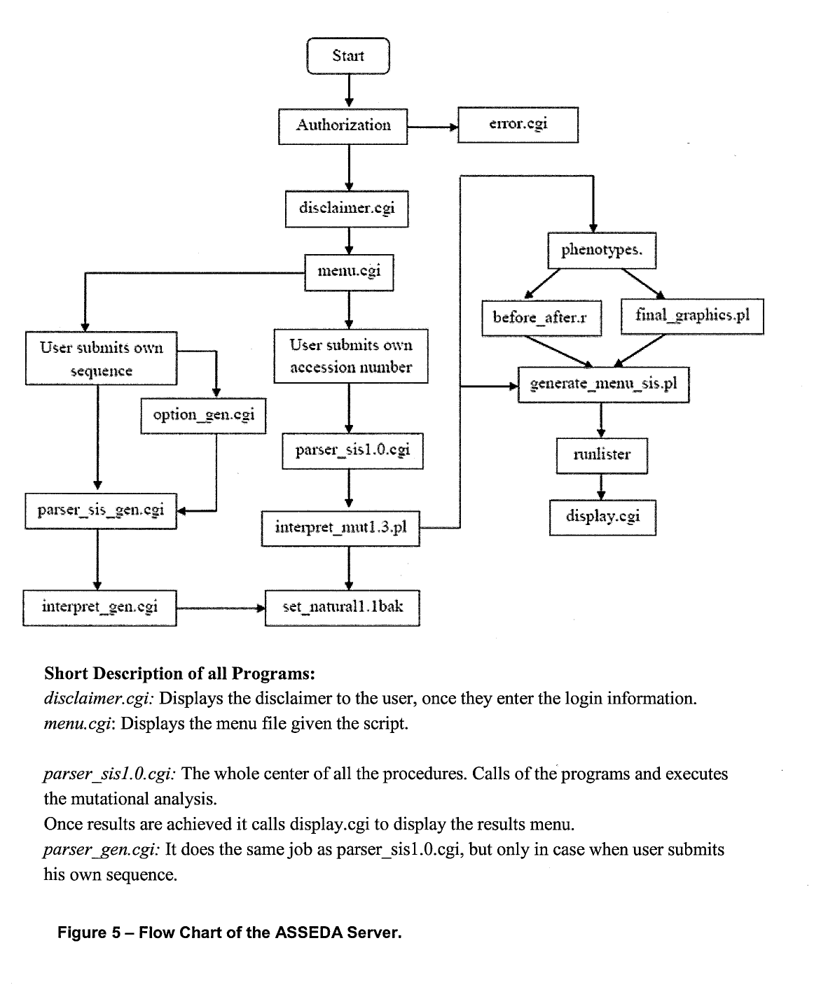

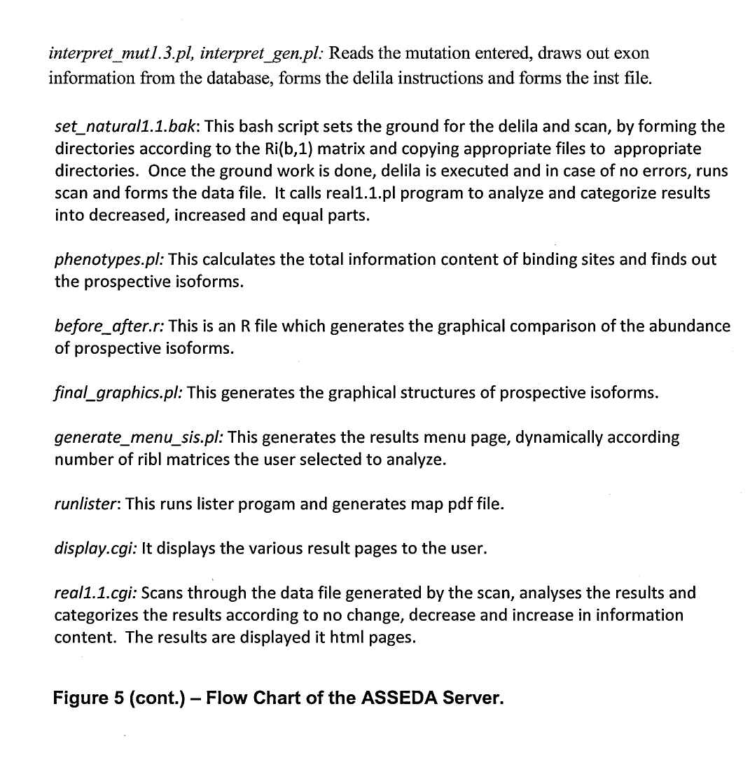

The ASSEDA server retains ASSA's capability to analyze changes in individual information content, but also predicts molecular phenotypes based on changes in R_{i,total}. ASSEDA and ASSA use the same interface to input sequence variants: HUGO-approved gene symbols, HGVS mutation nomenclature, and dbSNP identifiers, sequence window range around the mutation coordinate, and selected weight matrices as input (FIG. 2a; (Nalla and Rogan, 2005)). Mutation syntaxes are then translated into equivalent Delila instructions (Schneider et al., 1984). The ASSEDA server contains a new option that allows analysis of either splice site information, molecular phenotype based on exon information, or both (for system architecture and program flow diagrams, see FIGS. 4 and 5). Upon submission of a mutation, a set of GenBank accession identifiers (ID) corresponding to mRNAs associated with the submitted gene is suggested. These IDs now include mRNAs in the NCBI Reference Gene Sequence database (http://www.ncbi.nlm.nih.gov/RefSeq/; RefSeq). The IDs are differentiated according to GenBank accessions (in green) and RefSeq ID's (in blue). The longest mRNA accession number is selected by default, and the genomic structure of each RefSeq accession is hyperlinked to the selected ID.

The window range is a primary determinant of the number of potential isoforms reported, since larger windows capture additional potential cryptic splice sites. The feasibility of exon formation is assessed by their R_{i,total} values, and by using rule-based filters to ensure that only likely isoforms are reported. These eliminate cryptic exons with misordered splice sites, overlapping donor and acceptor sites, internal exons less than 30 nt in length (Dominski and Kole, 1991), predicted splice isoforms with <1% of exon inclusion relative to the mutated, natural exon strength (ΔR_{i,total} between two isoforms <6.65 bits). The server highlights isoforms with negligible expression when their R_{i,total} values are at least 1 bit below that of the R_{i,total} of the mutated exon. Tabular results can be sorted by column and is paginated, which is particularly helpful for mutations in which numerous cryptic exons are predicted. All rows with potentially expressed isoforms are uncolored, but the wild type exon is indicated in red. Splice isoforms that either cannot be expressed or minor forms (<5% of the major expressed form) that would not be detectable experimentally are, by default, filtered out. Without filtering, rows containing non-functional or minimally expressed predicted isoforms are highlighted in distinct colors: (1) Exons with misordered splice sites (light blue), (2) Potential cryptic exons with lower R_{i,total} values than normal or mutated exon (≦1% predicted expression; pink). (3) Isoforms with both incorrect splice site order and have low R_{i,total} values (green). The minimum reportable R_{i,total} value may also be selected using horizontal sliding scale bar which filters out potential exons below this threshold.

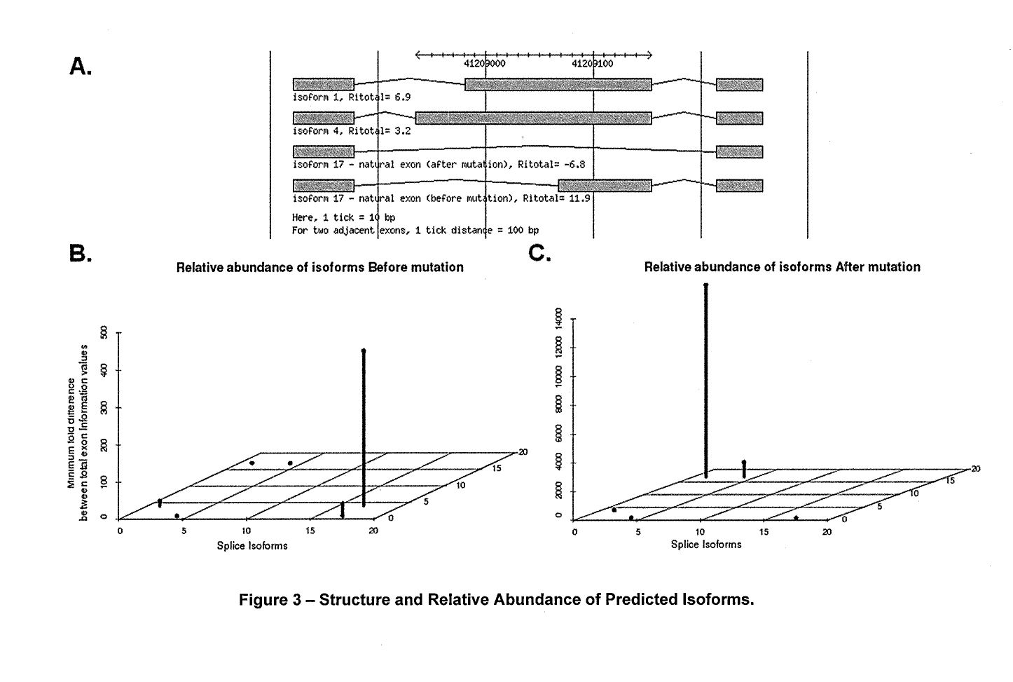

The server draws a set of box glyphs (FIG. 3a) depicting a set of exon structures and lengths of potential isoforms that are most likely to form exons. The index of each isoform and its R_{i,total} value are also indicated next to each structure as well as the approximate chromosome coordinates of the normal and cryptic exons.

The server also generates separate custom tracks of each isoform and uploads them to the UCSC genome browser, where they are displayed in the context of the exon containing the mutation as an embedded window within ASSEDA. Each isoform is spectrally color coded based on R_{i,total} content.

### Relative Abundance of Predicted Splice Isoforms

The server also displays pairwise differences in relative abundance for all predicted isoforms. The relative abundance or fold change in binding affinity of a single binding site is ≦2^{ΔRi}, where ΔRᵢ is the difference between the respective individual information contents of wild type and mutant type of the site (Schneider, 1997). We extend the idea of relative abundance of single binding site to multiple binding sites by comparing their R_{i,total} values. Suppose n and m are two alternative splice isoforms sharing at least one common splice site and their respective total information contents are R_{i,total(n)} and R_{i,total(m)}. If R_{i,total(n)}>R_{i,total(m)}, then the relative abundance of n over m will be ≦2^{ΔRi,total(nm)}, where ΔR_{i,total(nm)}=R_{i,total(n)}−R_{i,total(m)}. Relative transcript abundance is displayed as a multidimensional graph (with scatterplot3d, an R package for visualization of three dimensional multivariate data). The graph shows predicted pairwise differences in exon abundance (Z axis) of the X axis isoform relative to the one on the Y axis, both before (left graph) and after mutation (right graph). The isoform designations correspond to those shown in the other molecular phenotype tabs.

In order that the manner in which the recited and non-recited advantages and objects of the invention are obtained, a more particular description of the invention will be rendered by reference to specific embodiments thereof which are illustrated in the drawings. It is to be understood that these drawings depict only typical embodiments of the invention and are not therefore to be considered to be limiting of its scope, the invention will be described and explained with additional specificity and detail through the use of the accompanying drawings.

A brief description of the drawings are provided below to provide additional specificity and detail of the drawings.

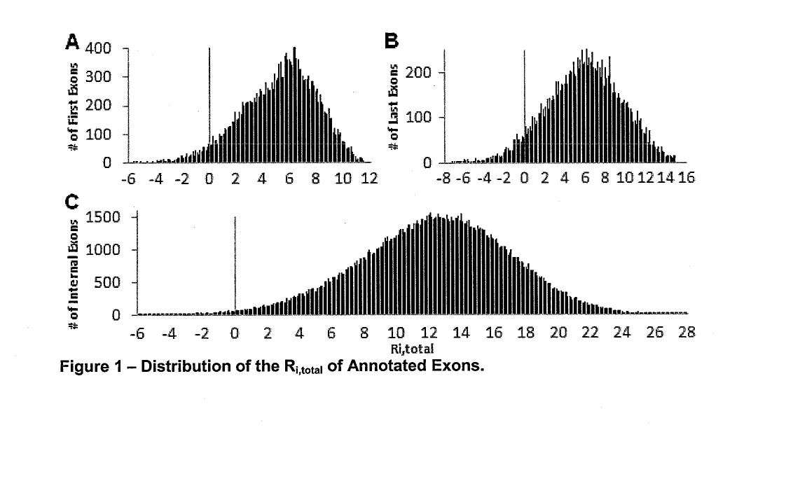

FIG. 1 shows distribution of the R_{i,total} of annotated exons. Distribution of the R_{i,total} of Annotated Exons. Histogram of R_{i,total} values for exons in the RefSeq database are illustrated for first (a), last (b), and internal exons (c). Nearly all internal exons exhibit total information contents exceeding zero bits (98.9%). The gap surprisal functions for first and last exons are not optimized for single splice site exons (4.7% and 7.0%, respectively, have R_{i,total} values below zero bits). The majority of false negative internal exons contain one or both splice sites that are either weak or are not recognized by either the U1- or U2 splicesomes.

FIG. 2 shows server input and results for BRCA1 mutation, chr17:g.412090680>A. A) User input. The window size of 200 nt increases the number of potential cryptic isoforms reported beyond the default length; B) Resulting table after applying splicing mechanism and exon abundance filters (isoforms 5-14 are not presented due to space limitations). The column headings show key binding site locations, initial and final values and changes in Rᵢ, as well as changes in R_{i,total}. The natural or mutated exon is listed in table row 17 (WT in legend below). Cells 1 and 4 (PI) indicate predicted cryptic isoforms with R_{i,total} values comparable or exceeding the strength of the natural exon (R_{i,total} final). Splice isoforms with R_{i,total}≦1 bit (>2 fold lower abundance; NE in legend) of the mutated natural exon are minimally expressed and filtered out. Rows 2 and 3 indicate predicted exons with misordered splice sites (NC), and rows 15 and 16 show exons which also would be minimally expressed (NC-NE); C) Only 3 of 35 potential isoforms are reported for the input mutation after filtering on these criteria.

FIG. 3 shows structure and relative abundance of predicted isoforms. Isoforms are depicted graphically according to their exon structures, relative abundance, and custom browser tracks in separate tabs. Isoform numbers in FIG. 3 refer to designations in FIG. 2c. Panels: (A) The scale above shows the genome coordinates of each of the isoforms. All prospective isoforms (sorted by R_{i,total}) are scaled according to their genomic coordinates (above glyphs). The exon skipping splice form is displayed for mutations where resulting R_{i,total}<0 bits; (B and C) Plots indicating predicted pairwise (x,y axes) relative minimum fold differences in abundance (z axis) of each isoform both before and after changes in R_{i,total} due to the mutation. Results are depicted for BRCA1, chr17:g.41209068G>A. Panel B shows that the natural wildtype exon (isoform 17) has the highest level of expression. After the mutation (Panel C), isoform 1, which activates a downstream cryptic splice site, is expected to be the dominant splice form. Note that the scale of the Z-axis will change between the panels, depending on the range of ΔR_{1,total} values resulting from the mutation.

FIG. 4 shows architecture of the ASSEDA server.

FIG. 5 shows flow chart of the ASSEDA server. The program flow chart of the server, with brief descriptions of the programs listed.

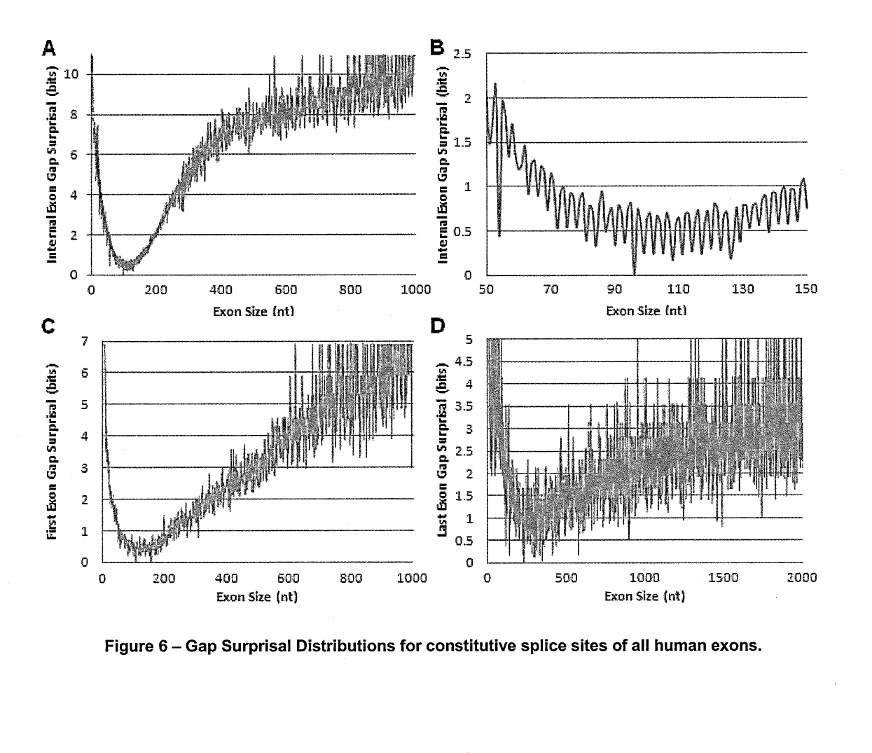

FIG. 6 shows Gap Surprisal distributions for constitutive splice sites of all human exons. The gap surprisal distribution is computed from the length and frequency of all exons in the genome (see methods). The length is based on the set of distances between the constitutive donor to acceptor. The results are truncated in the Figure to indicate distributions for exons ≦2000 nt in length. The gap surprisals are separated by category of exon: internal (panel A), first (panel C) and last (panel D) exons of genes. To illustrate the apparent triplet periodicity of the gap surprisal function associated with open reading frames in exons of common length (50-150 nt), we include panel B. Exons were extracted from the RefSeq database at the National Center for Biotechnology Information (http://www.ncbi.nlm.nih.gov/RefSeq/).

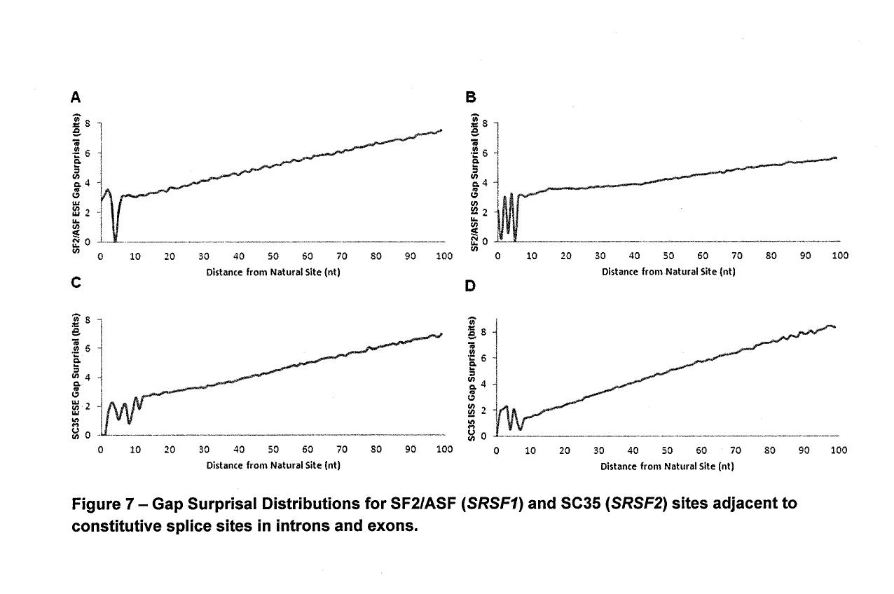

FIG. 7 shows Gap Surprisal distributions for SF2/ASF (SRSF1) and SC35 (SRSF2) sites adjacent to constitutive splice sites in introns and exons. Gap surprisal function distributions were derived for splicing regulatory sequences from the inter-site distance (nt) between all predicted sites of one type (either SC35 or SF2/ASF site) to the nearest constitutive splice site (either donor or acceptor). These distributions are computed separately for intron and exon locations of splicing regulatory sequences. The gap surprisal term and the Rᵢ value of the corresponding site are added to the other elements of R_{i,total}. The contributions of these terms (ie. their signs) are assigned based on whether a binding site is treated as an ISS(Rᵢ<0; g(L_{pq})>0) or as an ESE (Rᵢ>0; g(L_{pq})<0). The gap surprisal distributions are displayed for SF2/ASF exonic (A); SF2/ASF intronic (B); SC35 exonic (C); SC35 intronic (D). The windows are truncated at exons 100 nt in the images, however the software computation spans all possible inter-site lengths. A constant value is added to the computed gap surprisal to normalize the values so that the most common intersite distances are not penalized. For SF2/ASF, the most frequent exonic location was at position +4 relative to the splice site (normalization constant: 2.54 bits) and intron location was at position −2 (normalization constant: 3.25 bits). For SC35, the highest frequency exonic location was at position +1 (normalization constant: 3.40 bits) and intronic location was at position −1 (normalization constant: 3.33 bits).

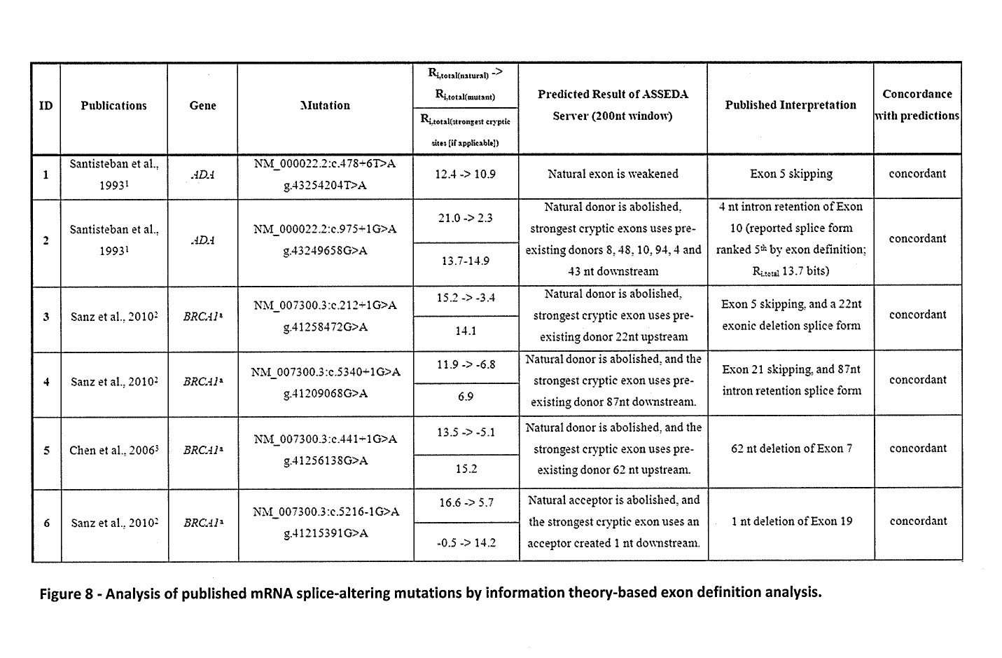

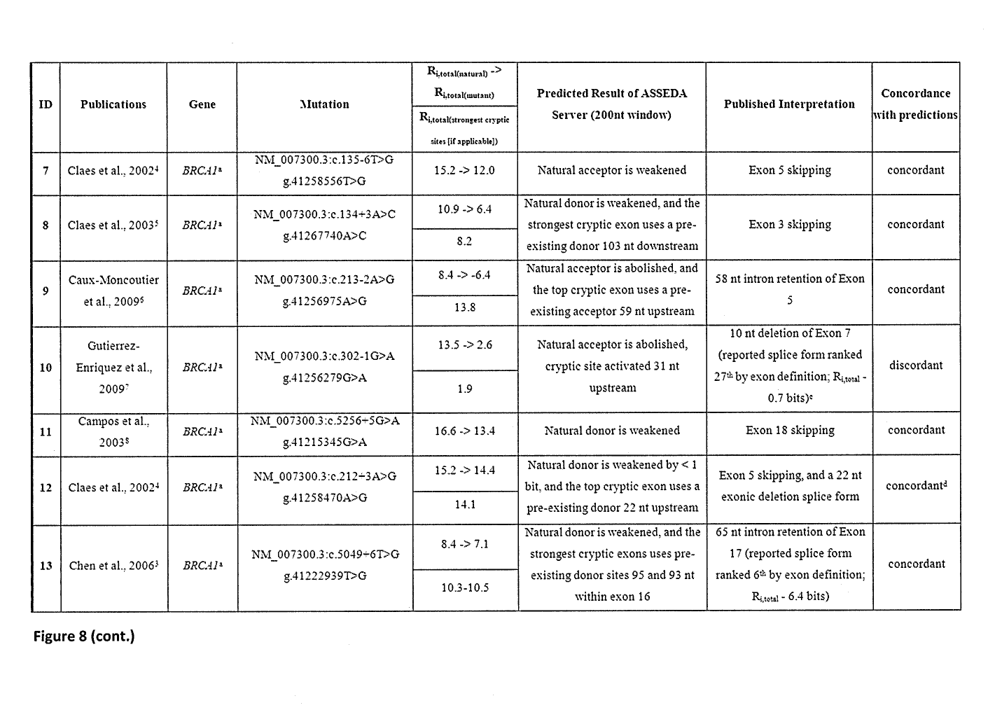

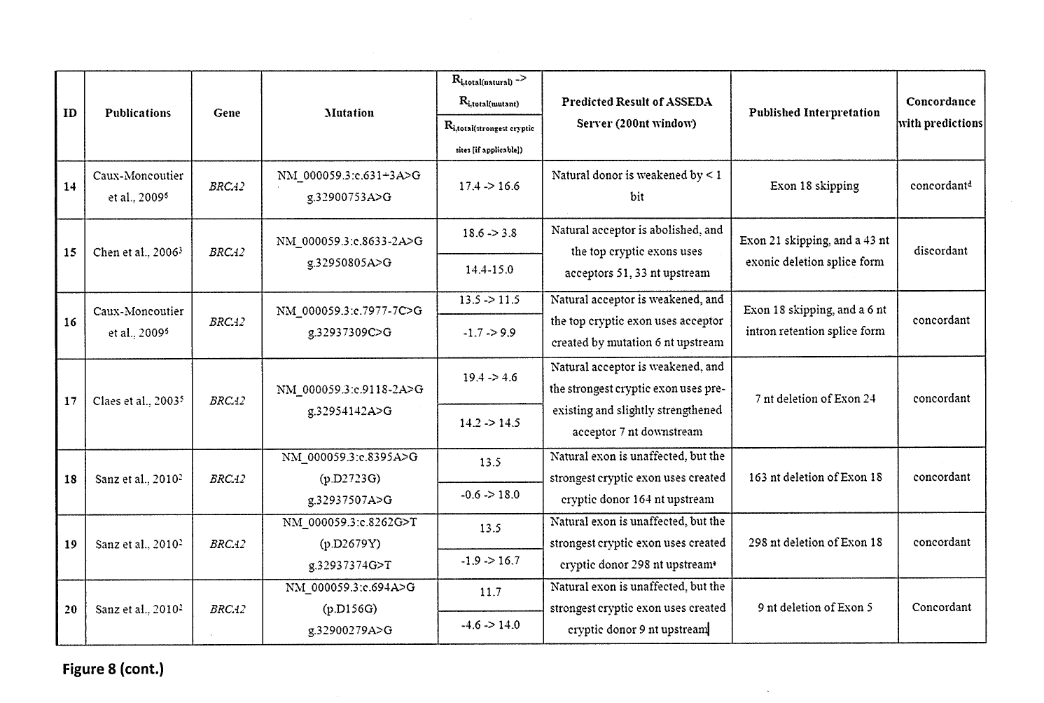

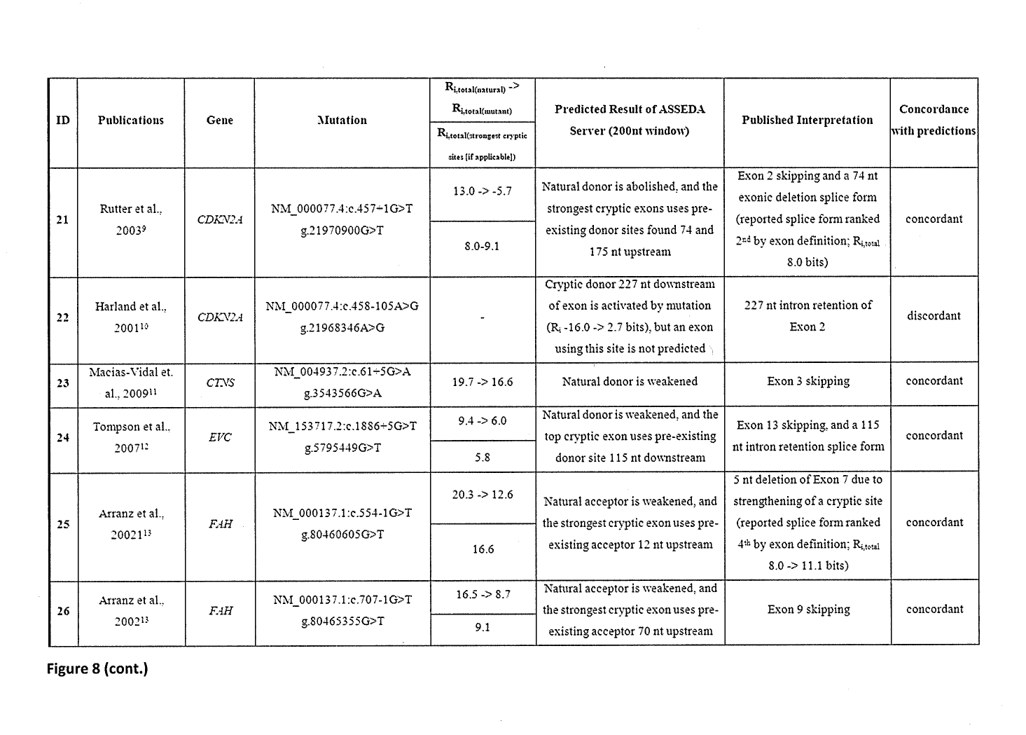

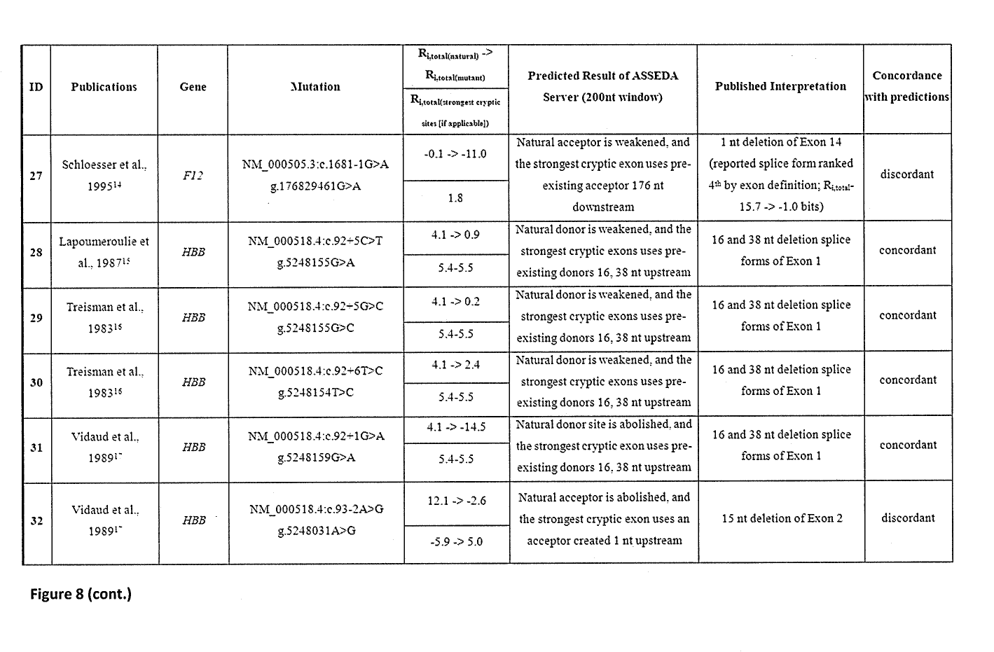

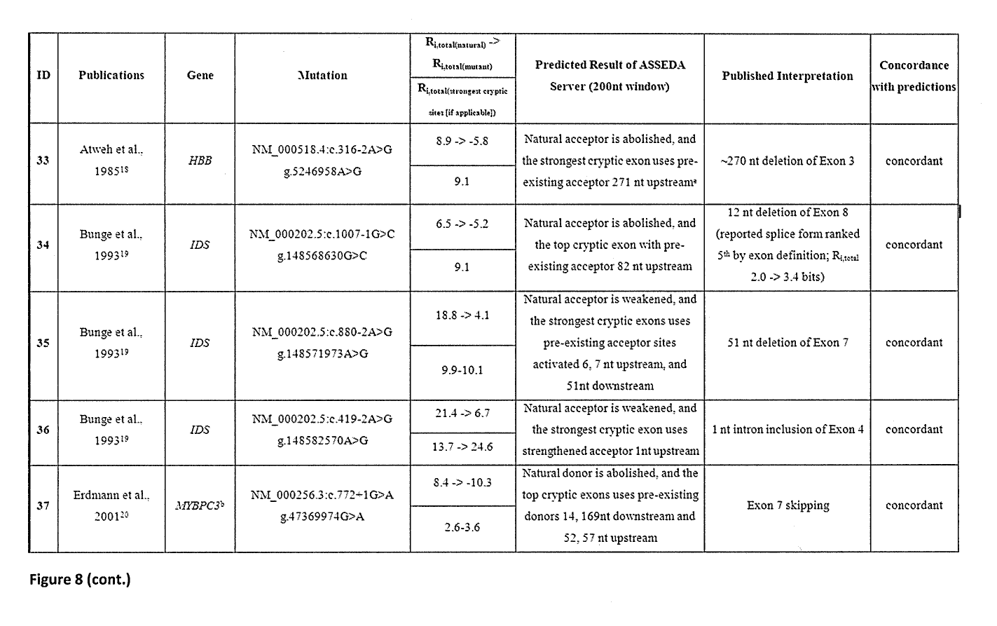

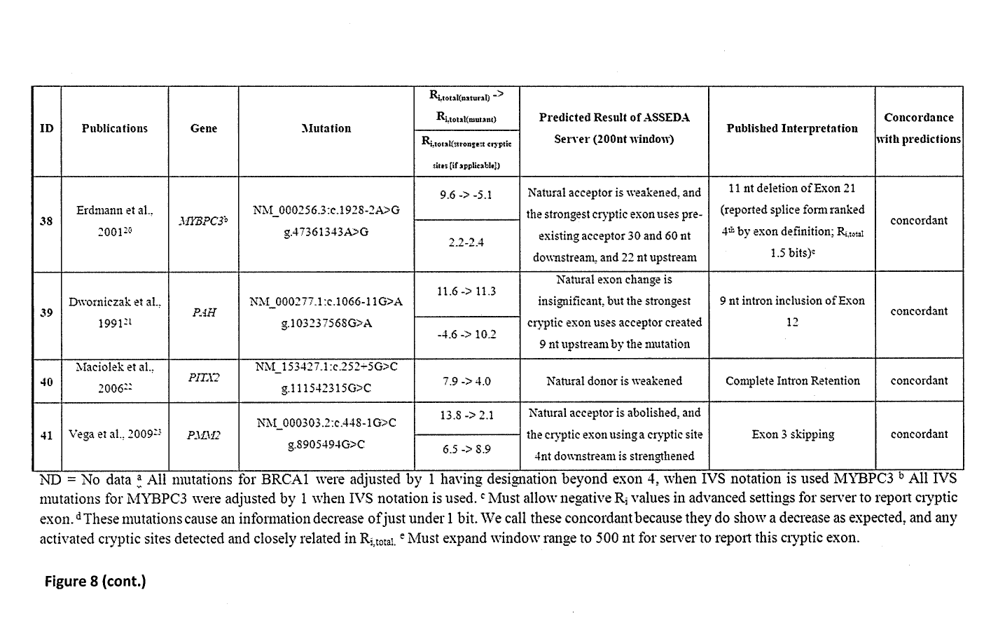

FIG. 8 shows analysis of published mRNA splice-altering mutations by information theory-based exon definition analysis. Published mutations known to affect mRNA splicing in various genes were analyzed using information theory based exon definition analysis. Mutations are given in both HGVS g. and c. format (c. format is mRNA dependent; position 1 is the A of the start codon). The ΔR_{i,total} values of mutations of the natural exon resulting from that mutation (as well as potential cryptic exons) are shown in the adjacent column. Interpretations of mutant exons predicted by ASSEDA relative to the published results are also reported. ND=No data^{a} All mutations for BRCA1 were adjusted by 1 having designation beyond exon 4, when IVS notation is used MYBPC3^{b} All IVS mutations for MYBPC3 were adjusted by 1 when IVS notation is used.^{c} Must allow negative Rᵢ values in advanced settings for server to report cryptic exon.^{d} These mutations cause an information decrease of just under 1 bit. We call these concordant because they do show a decrease as expected, and any activated cryptic sites detected and closely related in R_{i,total}.^{e} Must expand window range to 500 nt for server to report this cryptic exon.

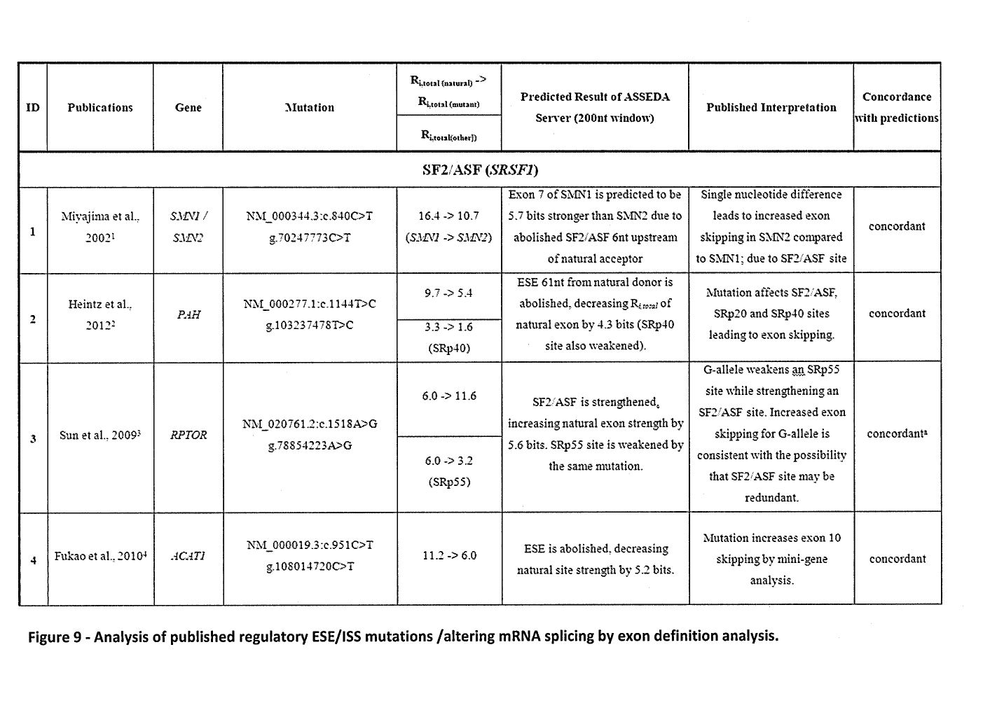

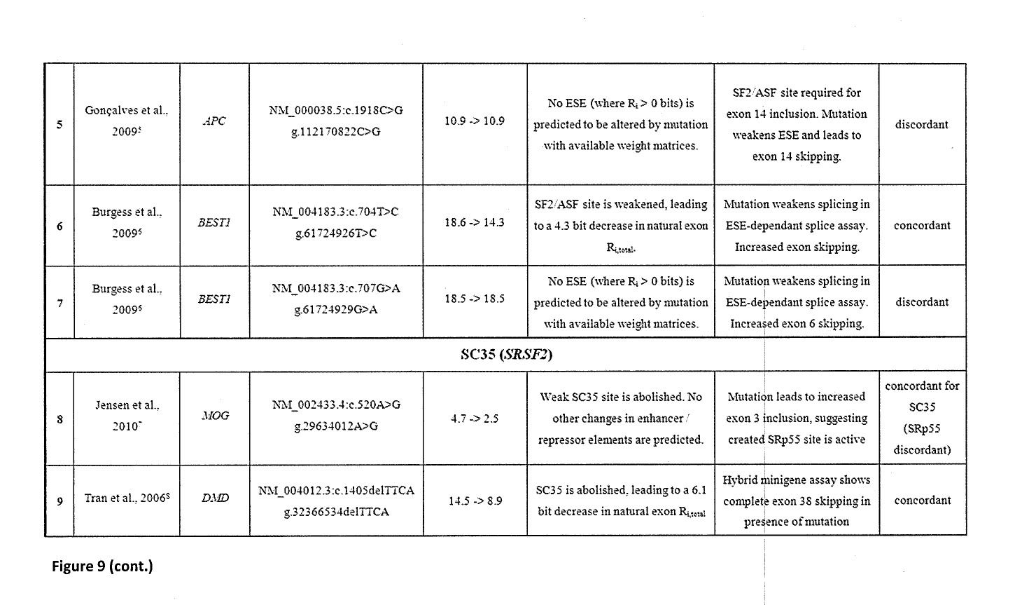

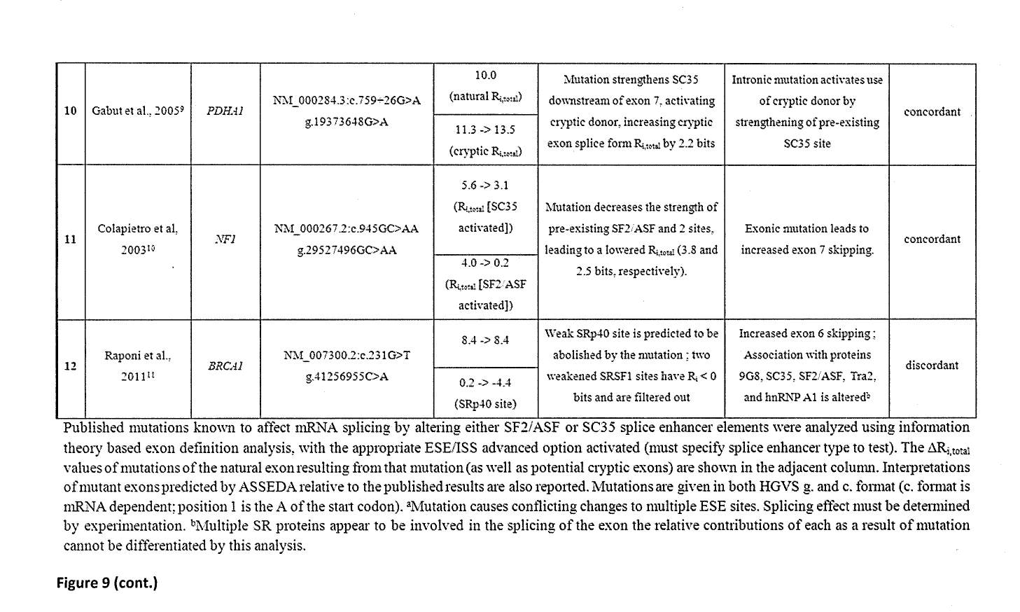

FIG. 9 shows analysis of published regulatory ESE/ISS mutations altering mRNA splicing by exon definition analysis. Published mutations known to affect mRNA splicing by altering either SF2/ASF or SC35 splice enhancer elements were analyzed using information theory based exon definition analysis, with the appropriate ESE/ISS advanced option activated (must specify splice enhancer type to test). The ΔR_{i,total} values of mutations of the natural exon resulting from that mutation (as well as potential cryptic exons) are shown in the adjacent column. Interpretations of mutant exons predicted by ASSEDA relative to the published results are also reported. Mutations are given in both HGVS g. and c. format (c. format is mRNA dependent; position 1 is the A of the start codon). ^{a}Mutation causes conflicting changes to multiple ESE sites. Splicing effect must be determined by experimentation. ^{b}Multiple SR proteins appear to be involved in the splicing of the exon the relative contributions of each as a result of mutation cannot be differentiated by this analysis.

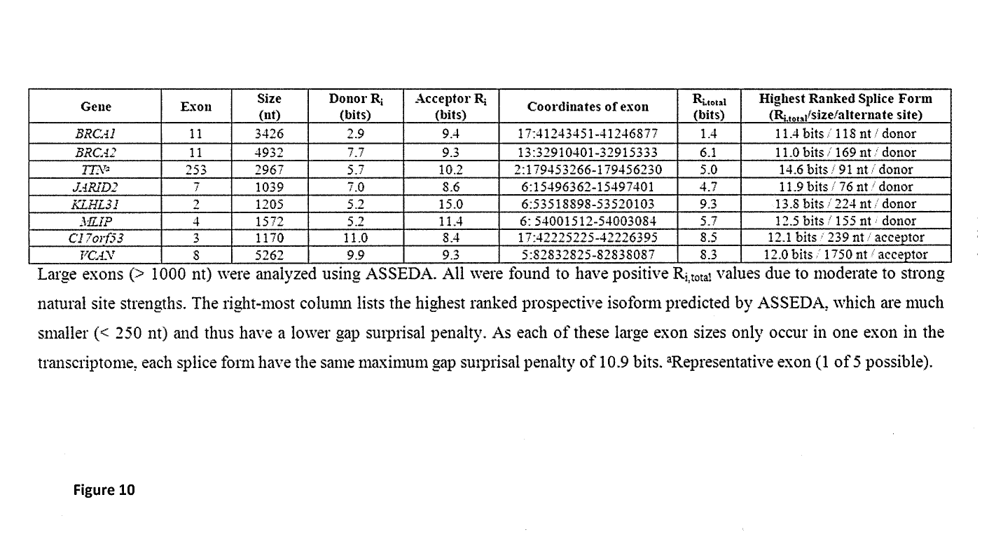

FIG. 10 shows analysis of normally spliced large (>1000 nt) exons. Large exons (>1000 nt) were analyzed using ASSEDA. All were found to have positive R_{i,total} values due to moderate to strong natural site strengths. The right-most column lists the highest ranked prospective isoform predicted by ASSEDA, which are much smaller (<250 nt) and thus have a lower gap surprisal penalty. As each of these large exon sizes only occur in one exon in the transcriptome, each splice form have the same maximum gap surprisal penalty of 10.9 bits. ^{a}Representative exon (1 of 5 possible).

FIG. 11 shows sequence logo and weight matrix of splicing regulatory sequence binding sites. Information-based position weight matrices were generated using SELEX (Liu et al., 1998) sequences, as well as the sequences of other sites confirmed in published binding studies. Left: sequence logo with error bars indicating 1 standard deviation. Right: information weight matrix (Rᵢ,(b,l)).

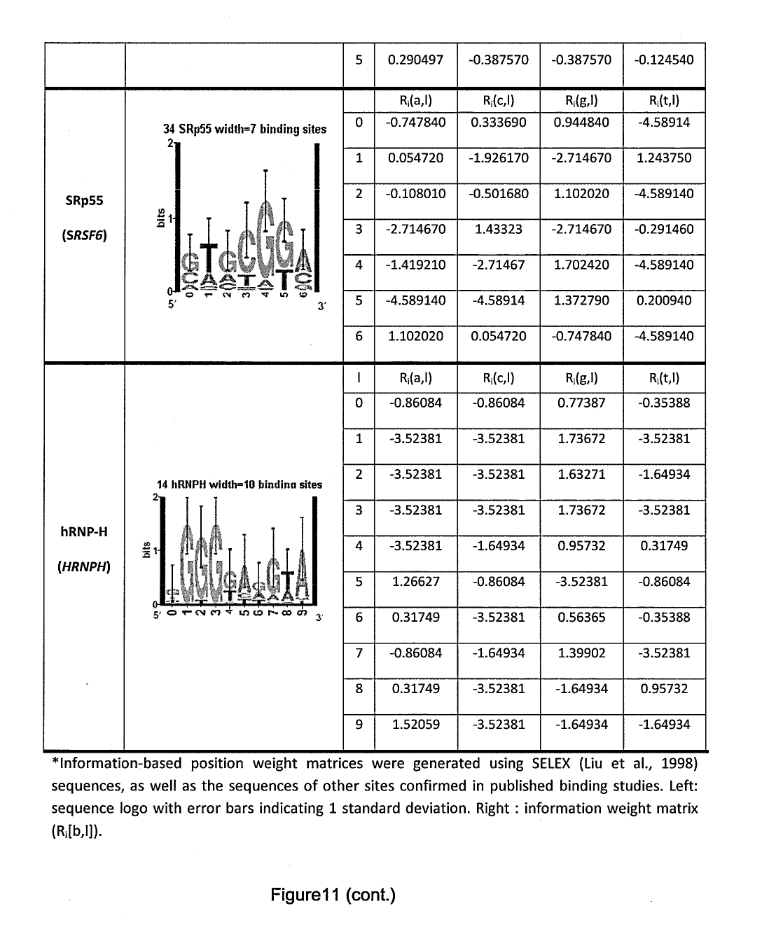

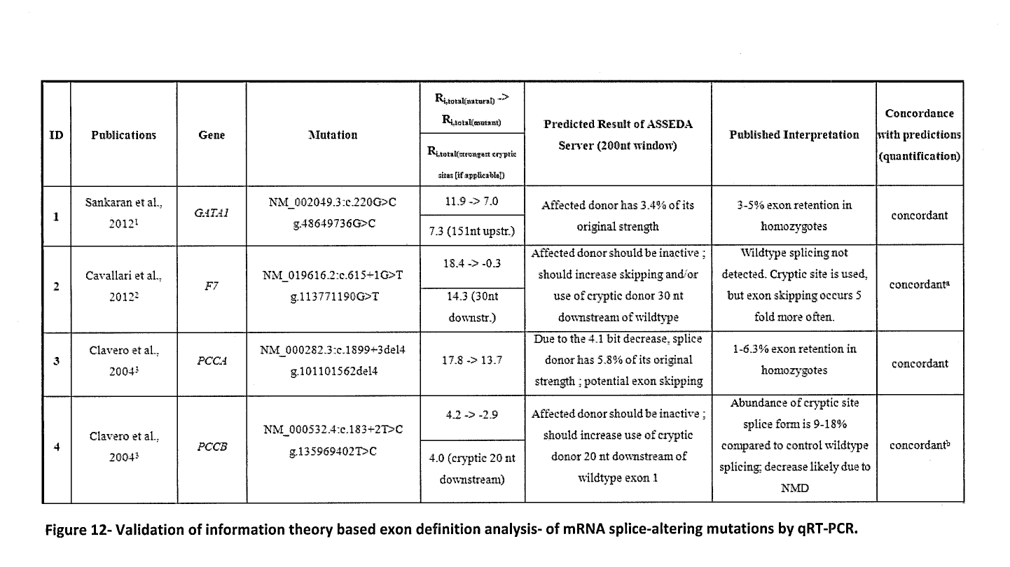

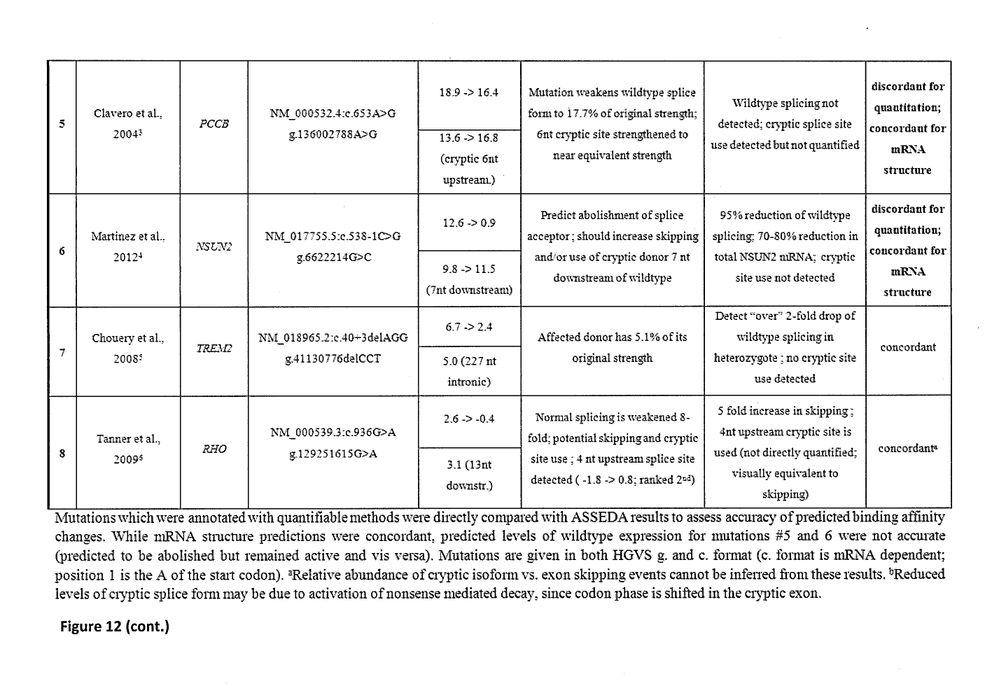

FIG. 12 shows validation of information theory based exon definition analysis-of mRNA splice-altering mutations by qRT-PCR. Mutations which were annotated with quantifiable methods were directly compared with ASSEDA results to assess accuracy of predicted binding affinity changes. While mRNA structure predictions were concordant, predicted levels of wildtype expression for mutations #5 and 6 were not accurate (predicted to be abolished but remained active and vis versa). Mutations are given in both HGVS g. and c. format (c. format is mRNA dependent; position 1 is the A of the start codon). ^{a}Relative abundance of cryptic isoform vs. exon skipping events cannot be inferred from these results. ^{b}Reduced levels of cryptic splice form may be due to activation of nonsense mediated decay, since codon phase is shifted in the cryptic exon.

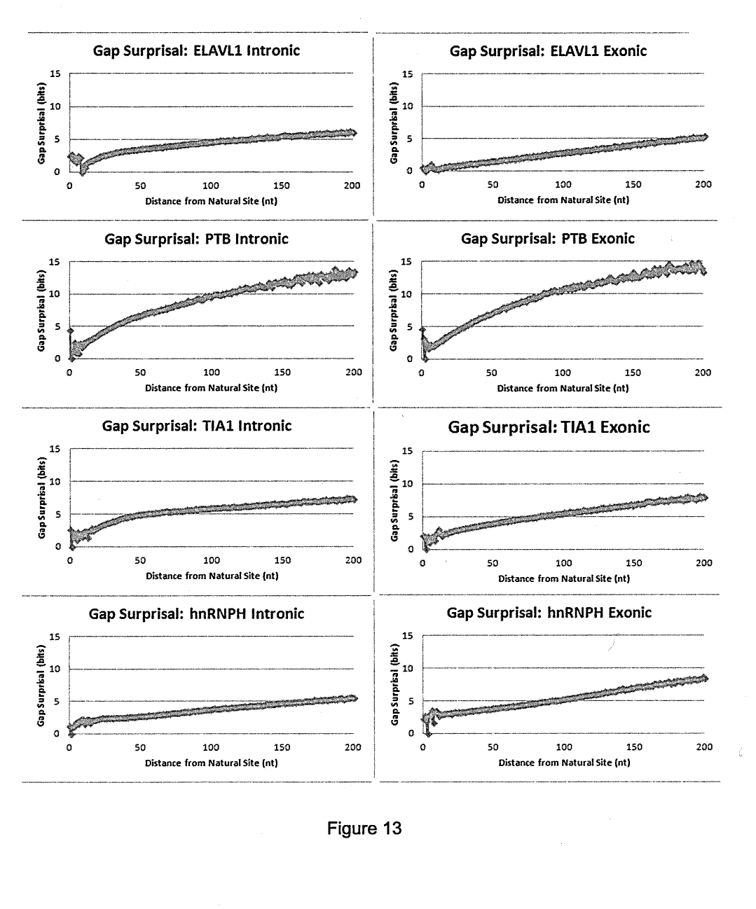

FIG. 13 shows the gap surprisal distributions for ELAVL1, PTB, TIA1 and hnRNPH.

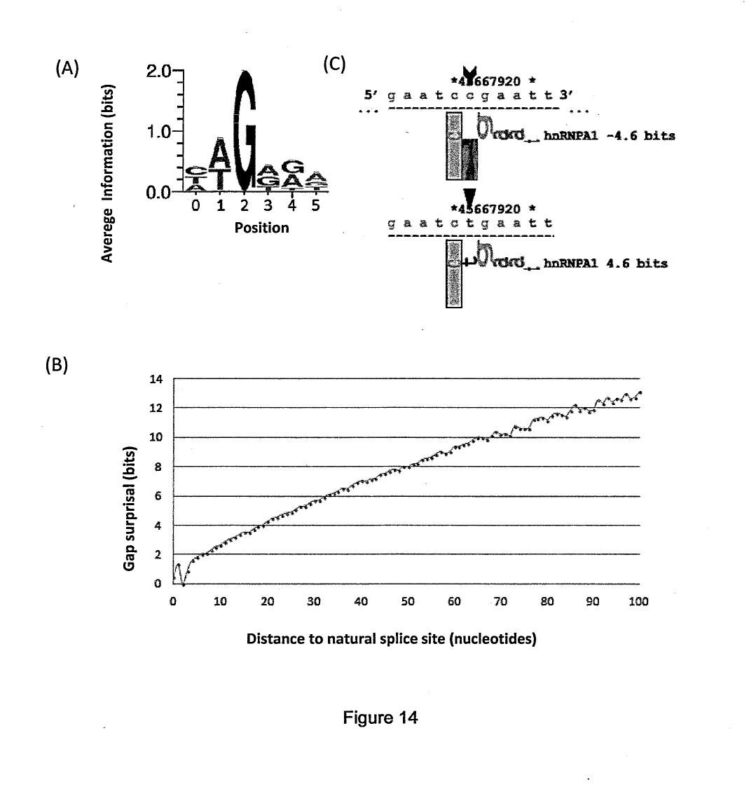

FIG. 14 shows hnRNP A1 binding site and description of information theory-based model. Panel (A) The opal codon in FANCM contained the core sequence of the novel hnRNP A1 site (Sequence logo, positions 1-3). This binding site sequence is frequently present in sites crosslinked to hnRNP A1 protein (Huelga et al. 2012); (B) The gap surprisal function for hnRNP A1 binding sites shows that sites within exons become significantly less frequent as their distance from the natural splice site increases. This is consistent with role of hnRNP A1 as an exon splicing silencer element, promoting exon skipping. See Olsen et al., Human Mutation, Volume 35, Issue 1, pages 86-95 (2014). hnRNP A1 binding sites is or close to the exon boundary in order to proofread U2AF binding at the 3′ splice site (Tavenez et al. 2012); otherwise, definition of the exon is abrogated; (C) Sequence walkers depicting the creation of a novel 4.6 bit hnRNP A1 binding motif spanning positions 45667919-45667925.

The following examples are provided for purposes of illustration of embodiments of the present disclosure only and are not intended to be limiting. The reagents, chemicals, instruments and other materials are presented as exemplary components or reagents, and various modifications may be made in view of the foregoing discussion within the scope of this disclosure. Unless otherwise specified in this disclosure, components, reagents, protocol, and other methods used in the disclosure, as described in the Examples, are for the purpose of illustration only.

### Example 1

### Exon Definition by Information Analysis of Functional Exons

Gap surprisal values of all exon lengths were determined from their respective frequencies in the exome of all RefSeq genes. The gap surprisal penalty was then normalized so that the most common internal exon length (96 nt; n=172,250) was zero bits, by subtracting a constant value of 6.59 bits (its log₂ frequency). Less frequent exon lengths were scaled to this value by subtracting this constant from their respective gap surprisal values. First and terminal exons are, respectively, missing either a donor or an acceptor splice site, and exhibit a broader range of exon lengths. Separate gap surprisal distributions were computed for these exons. The most frequent first and last exons were, respectively, 158 (n=23,471) and 232 (n=21,261) nt in length, corresponding to gap surprisals of 7.8 and 9.4 bits, respectively. R_{i,total} values were >0 bits for 98.9% of internal exons, 95.3% of first exons, and 93.1% of last exons (FIG. 1). Although inclusion of the gap surprisal term resulted in fewer false positive splice isoforms (Robberson et al., 1990; Dominski and Kole, 1992), a slightly higher proportion of first and last exons had negative R_{i,total} values. Since most of these splice sites in these exons exhibited positive Rᵢ values (72% of first, 87% last exons), the negative R_{i,total} values may be the result of other unknown factors contributing to recognition of these exons not accounted for, or to suboptimal gap surprisal functions.

### Example 2

### Interpretation of Splicing Mutations by Exon Definition Analysis

To assess whether the proposed model of exon definition produced results consistent with observed mutant spliced products, we evaluated a series of reported splicing mutations for which end-point (FIG. 8) and quantitative (FIG. 12) expression studies had been performed. A typical molecular phenotypic prediction is indicated in FIG. 2 (BRCA1 IVS20+1G>A or HGVS designation chr17: g.41209068C>T; FIG. 8, Mutation #4). The tabular results indicate genomic coordinates of donor and acceptor sites, their relative distance from the closest natural site, and the change in Rᵢ for these sites. Each row indicates R_{i,total} both before and after mutation for a different set of exon boundaries corresponding to a distinct predicted isoform. Predicted isoforms are sorted according to these values, whose fold differences in binding affinity are ≦2^{ΔRi,total} (Schneider, 1997).

Initially, 20 potential isoforms are found for this mutation, of which those with the highest R_{i,total} values and the affected natural exon are indicated (FIG. 2b). Based on the mechanism of exon recognition and the ΔR_{i,total} values, only a subset of these indexed isoforms is likely to be expressed. Splice site polarity is specified such that a functional acceptor splice site cannot occur downstream of a natural donor splice site to define an exon, and vice versa (Berget, 1995). The server eliminates exons with misordered splice sites, removing many false positive splice isoforms which do not conform to the natural mRNA splicing mechanisms. Pairs of splice donor and acceptor sites that either overlap each other are also not considered as potential exons (Nalla and Rogan, 2005; Robberson et al., 1990). Predicted low abundance natural and cryptic isoforms with undetectable expression (FIGS. 2b and 2c) are also filtered out.

The structures and lengths of each potential isoform (natural, cryptic, skipped) are also displayed in a separate tab (FIG. 3a). The central exon affected by the mutation is drawn to scale, however flanking intron sequences are condensed for presentation. In the example above, the exon 20 donor site in chr17: g.41209068C>T (R_{i,total} 11.9->−6.6 bits) is inactivated and an corresponding isoform with exon skipping is shown. The relative abundance (Z axis) of different pairs of indexed isoforms (X and Y) before (FIG. 3b) and after (FIG. 3c) mutation also predicts a number of cryptic isoforms. Isoform 1 uses a pre-existing donor 87 nt downstream that is at least 13,307 (i.e. ≦2^{13.7 bits}) fold more abundant than the mutated exon, but would not normally be detected because it is 32 fold) (≦2^{5.0}) less abundant than the normal exon. mRNA analyses have shown that this mutation results in both cryptic and skipped splice forms (Sanz et al., 2010), however isoform 4 which contains 133 of intronic sequence (FIGS. 2c and 3a), was not detected.

### Example 3

### Impact of ESE/ISS Elements

Elements recognized by splicing regulatory proteins, SF2/ASF, SC35, SRp40, SRp55, and hnRNP-H (HNRNPH1), can now be analyzed with ASSEDA, however these matrices are based on many fewer sites (usually <50), and the Rᵢ values may not be as accurate as constitutive splice sites, especially at the low end of the distribution. The server computes Rᵢ values of any of these individual sites and can incorporate mutations at either SF2/ASF or SC35 sites into the R_{i,total} computation. Since a mutation can affect multiple predicted sites, the site with the highest Rᵢ value altered by the mutation is analyzed, unless a second cryptic site is strengthened resulting in final Rᵢ is exceeding that of the original binding site.

A second gap surprisal function, based on the distances between known natural constitutive sites and the closest predicted splicing regulatory site of the same type, was also applied in the R_{i,total} calculation. Exonic (ESE) and intron (ISS) have independent gap surprisal distributions (FIG. 9). The ubiquity of these splicing regulatory sequences suggested that their predicted distributions would be biased towards shorter inter-site distances, however there were distinct preferences for certain distances. 17.2% of all exonic SF2/ASF sites were separated by 4 nt from a natural splice site (n=562,786; comparatively, all other distances between 0-10 nt range from 1.5-4.4% in frequency). The most common intronic SF2/ASF sites were 1, 3 and 5 nt from the natural site (9.3%, 7.1% and 10.5% respectively; n=562,788). The most common SC35 site inter-site exonic distances were 0, 4 and 7 nt (9.5%, 6.5%, 6.6% respectively) and intronic distances were spaced 1 and 2 nt from the splice site (9.9% and 9.5%). In all cases, frequency decreased with increased inter-site distance. The distribution of predicted SRp40 distances showed no distance bias; there was a gradual inverse relationship between frequency and distance from the natural site (maximum frequency was <0.1% of the sites).

To assess the effect of including SC35 and SF2/ASF sites in the exon definition model, we evaluated 12 reported mutations/variants in either SF2/ASF or SC35 sites that were reported to affect splicing at adjacent splice sites (FIG. 9). Eight of 12 predictions of ASSEDA were concordant with the published results (Supp. Table 4 mutations #1-4,6,9 and 11 are predicted to weaken splicing and lead to exon skipping; #10 strengthens an intronic SF2/ASF site and activates a cryptic donor). A single nucleotide difference between SMN1 and SMN2 (c.840C>T) is known to alter an SF2/ASF exonic site, resulting in skipping of exon 7 in SMN2 (Cartegni and Krainer 2002). The SF2/ASF variant in SMN2 reduces ΔR_{i,total} of exon 7 in SMN2 by 5.7 bits relative in SMN1, corresponding to a 52 fold difference in exon recognition, consistent with skipping of this exon in SMN2 (FIG. 9: #1).

### Example 4

### Analysis of Normally Spliced Large (>1000 nt) Exons

The exon definition models imply that rare exons (regardless of length) will have large gap surprisal penalties. This is supported by the fact that, for exons beyond a few hundred nucleotides, the penalty function is increases with length until it asymptotes at exon lengths present once in the genome. The significant gap surprisal penalties for long exons raise the question as to how well the model performs at the extreme lengths to correctly distinguish natural from decoy exons. The model fails if the contributions of the gap surprisal term exceed the Rᵢ values of both natural splice sites. In fact, this is generally not the case.

To assess the ability of the server to predict naturally occurring large exons, 8 large internal exons in genes BRCA1-ex11, BRCA2-ex11, TTN-ex253, JARID2-ex7, KLHL31-ex2, C6orf142-ex4 (MLIP), VCAN-ex8 and C17orf53-ex3 were evaluated using ASSEDA (FIG. 10). Despite the large (>10 bit) gap surprisal penalties, the R_{i,total} values for each of these exon was still exceeded 0 bits. This can be attributed to their strong donor and acceptor sites, which appear to be essential for large exon recognition ((Bolisetty and Beemon, 2012); the exception being the donor site of BRCA1 exon 11 (2.9 bits)). These predicted shorter splice forms are present in BRCA1 mRNA, however they do not encode full length protein. For example, the highest ranked prospective isoform for BRCA1-ex11 was a 118 nt long alternate splice form (NM_{—}007298.3). These large exons were not ranked first, as the R_{i,total} of smaller exons (<250 nt) tended to have higher overall R_{i,totals} (lower gap surprisal penalty). Larger exons tend to have a higher ratio of enhancers to repressors compared to smaller exons (Bolisetty and Beemon, 2012). This suggests that gap surprisal function will need to be refined, or contributions of other splicing regulatory proteins will need to be incorporated into R_{i,total} in order to correct the ranking of splice isoforms from long exons.

### Example 5

### Generation of Information Theory-Based Models of mRNA Splicing Regulatory Proteins

Successful implementation of the information theory-based exon definition model is dependent on the quality of the data used to create the information weight matrices that locate and define the strengths of binding sites. Splice junctions are precisely defined and experimentally validated.

CLIP-seq libraries for hnRNP A1 (Huelga et al., 2012), and other splicing regulatory binding sites were used to derived information-theory based position weight matrices (PWM). CLIP-seq libraries were generated by methods that chemically link an RNA binding protein to its cognate binding sites throughout the transcriptome, followed by antibody pull down of the protein crosslinked to these binding sites, then followed by conversion of RNA to cDNA in vitro, and preparation of libraries of many binding sites, and finally by high throughput DNA sequencing of the libraries. PoWeMaGen software, which uses Bipad (Bi and Rogan, 2004) to generate a minimum entropy alignments, generates a series of potential binding site models over a range of input parameters. To mitigate against phasing the alignment on natural splice sites instead of adjacent hnRNP A1 binding sites, models were built from shorter sequences, ranging in lengths from 18-25 nt. The optimal model was determined by maximizing incremental information by varying binding site length (6-10 nt), number of Monte Carlo cycles (250-5000), and allowing either zero or only one site per sequence (OOPS). The model with the highest average information used a maximum fragment length of 18 nt, 1000 Monte Carlo cycles, OOPS, and a single block binding site length of 6 nt.

CLIP-seq data were used to compute PWMs for the following RNA binding proteins that participate in the mRNA splicing reaction and/or in exon definition:

### T1A1

### Ri(b,l) Length of PWM—12 nt

Monte Carlo cycles—1000
ZOOPS (Zero Or One site Per Sequence)—On

### Source:

Wang Z, Kayikci M, Briese M, Zarnack K, Luscombe N M, Rot G, Zupan B, Curk T, Ule J. iCLIP predicts the dual splicing effects of TIA-RNA interactions. PLoS Biol. 2010 Oct. 26; 8(10):e1000530

### PTB

### Ribl Length—6 nt, 10 nt

Monte Carlo cycles—250, 1000

### ZOOPS—On, On

### Source:

Xue Y, Ouyang K, Huang J, Zhou Y, Ouyang H. Li H, Wang G. Wu Q, Wei C, Bi Y, Jiang L, Cal Z, Sun H, Zhang K, Zhang Y, Chen J, Fu X D. Direct conversion of fibroblasts to neurons by reprogramming PTB-regulated microRNA circuits. Cell. 2013 Jan. 17; 152(1-2):82-96.

### HuR

### Ribl Length—7 nt

Monte Carlo cycles—250
ZOOPS—Off (ON ribl is also available, but is very similar)

### Source:

### Kishore S, Jaskiewicz L, Burger L, Hausser J, Khorshid M, Zavolan M.

A quantitative analysis of CUP methods for identifying binding sites of RNA-binding proteins. Nat. Methods. 2011 May 15; 8(7):559-64.

Each model or PWM was validated with a set of independently published binding sites and if available, mutations in those binding sites. As an example, validation of hnRNP A1 binding sites and mutations are presented, however the same approach was used for the other PWMs. A coding sequence mutation in the ETFDH gene c.158A>G creates a 5.9 bit hnRNP A1 site and increases exon skipping. See Olsen et al. (2014). BRCA2 mutation c.8165C>G similarly increases skipping and is predicted to create a 6.2 bit site (Liede et al., 2002). In contrast, the variant c.1161A>G in ACADM decreases exon skipping of exon 11 by reducing the strength of an hnRNP A1 site (6.1 to 1.4 bits). The model also predicted the existence of two strong hnRNP A1 binding site in a region of ATM shown to bind to the splicing regulator (Pastor and Pagani, 2011).

The effects of mutations at hnRNP A1 sites on exon definition were determined from the total information content (R_{i,total}) by incorporating changes in the strengths of these sites, corrected for the gap surprisal, which represents the distance between the hnRNP A1 site and the natural splice site. Gap surprisal values were determined by scanning the genome for hnRNP A1 sites with the PWM, and then determining the frequency of each interval length between known natural sites and the nearest hnRNP A1 site, separately for exons and introns. Differences between the natural and mutated exon R_{i,total} values correspond to changes in the abundance of the respective isoforms, and can predict exon skipping. The calculation is carried out by the Automated Splice Site and Exon Definition Analysis Server (ASSEDA; http://splice.uwo.ca); See Mucaki et al. Prediction of Mutant mRNA Splice Isoforms by Information Theory-Based Exon Definition. Hum Mutat. 34:557-65 (2013), which is hereby incorporated by reference into this disclosure. Exon definition analysis in ASSEDA was validated for a set of mutations that affect hnRNP A1 binding site strength. BRCA2 variant c.8165C>G decreases the R_{i,total} from 13.5 to 3.2 bits and results in exon skipping. ACADM variant c.1161A>G, which reduces exon skipping, increases the R_{i,total} from 18.5 to 20.1 bits.

Table 1 summarizes the validation results for models derived CLIP Seq data by evaluating published, peer reviewed binding sites in individual genes.

TABLE 1
Summary of validation results

| RNA | Binding |
|---|---|
| binding | sites |
| protein | Validated |
| 9G8 | 1 of 4 |
| TIA1 | 7 of 7 |
| PTB | 4 of 4 |
| HuR | 6 of 6 |
| hnRNPA1 | 3 of 3 |
| hnRNPC | 3 of 4* |
| hnRNP | 0 of 1 |
| A2/B1 | |
| hnRNP F | 1 of 2 |
| hnRNP U | 1 of 1 |

Valation of the model is measured by the success rate of binding site models to predict published binding sites in the sequence interval described in the literature publication (successfully detected sites vs total number of binding sites tested). The exact location for the binding site was not always known from the publication, and in those cases, we sought to detect the strongest sites with the highest Ri values within that region, as described below. The results of optimal model construction include sequences logos and Ri(b,l) matrices, and links to the papers reporting the binding sites, among others.

Based on these validation results, the PTB and hnRNP A1 models have been qualified for mutation analysis. The information contents generated from these PWMs are completely concordant with the published results for all known binding sites, and their motifs (as depicted by the corresponding sequence logos) have a distinct, complex pattern.

The TIA1, HuR and hnRNP C model validation was also quite successful, but these PWMs consist of low complexity, T-rich motifs (based on DNA sequence, in RNA, which the protein binds to, these are Uridine) that have lower specificity than the PTB and hnRNP A1 binding sites. For TIA1 and HuR, this pyrimidine-rich region is where binding is expected. There have been concerns that these models will positively identify a binding site in nearly any poly-T rich region. As an example, one can refer to the HuR model, in which almost all information is derived from poly-T.

Summary of data on RNA binding protein motifs that are involved in mRNA splicing obtained by entropy minimization of Clip-Seq data is provided in the following text.

TIA1/TIAL1

TIA-1 promotes U1 snRNP binding to the 5′ splice site of intron 6 of FAS. Exonic TIA-1 binding to Uridine-rich sequences mediate repression by PTB at the acceptor (3′) site, promoting exon skipping (JoséMaria Izquierdo, Nuria Majós, Sophie Bonnal, Concepción Martínez, Robert Castelo, Roderic Guigó, Daniel Bilbao, Juan Valcárcel, Regulation of Fas Alternative Splicing by Antagonistic Effects of TIA-1 and PTB on Exon Definition, Molecular Cell, Volume 19, Issue 4, 19 Aug. 2005, Pages 475-484). This model does correctly recognize exon 3′ terminus at position 573, 3.2 bit site at 576, 4.9 bit site at 596, and a 3-4 bit cluster from 600-602.

The RNA-binding protein TIA-1 preferentially enhances the use of 5′ splice sites linked to IAS1 (for example, the alternative K-SAM exon in FGFR2 gene)—which are then activated by overexpression of TIA1. See Del Gatto-Konczak F, Bourgeois C F, Le Guiner C, Kister L, Gesnel M C, Stévenin J, Breathnach R. The RNA-binding protein TIA-1 is a novel mammalian splicing regulator acting through intron sequences adjacent to a 5′ splice site. Mol Cell Biol. 2000; 20(17):6287-99.

Approximately 20 nucleotides beyond the end of the K-SAM exon, information analysis predicts large cluster of strong binding sites (chromosome 10:123278160-123278310), associated with a long polyT/poly A track. This result is consistent with the well described property of TIA-1 binding to polyAU-rich domains of RNA.

| Chr. Coord. | Ri value |
|---|---|
| 123278167 | 5.669410 |
| 123278168 | 10.217979 |
| 123278169 | 2.813830 |
| 123278170 | 5.144820 |
| 123278171 | 4.534150 |
| 123278172 | 8.654270 |
| 123278173 | 1.410610 |
| 123278177 | 4.872140 |
| 123278178 | 1.938000 |
| 123278179 | 5.716410 |

In the SMN2 gene, exon 7 inclusion is regulated by TIA-1 interacting with the U1 SNRNP. See N. Singh and R. Singh, Alternative splicing in spinal muscular atrophy underscores the role of an intron definition model, RNA Biol. 2011 July-August; 8(4): 600-606. There are two validated TIA-1 sites within the interval (chr5:69,372,420-69,372,490).

| Chr. Coord. | Ri value |
|---|---|
| 69372436 | 6.438010 |
| 69372437 | 1.917100 |
| 69372438 | 3.805560 |
| 69372439 | 4.751070 |
| 69372441 | 2.209620 |
| 69372456 | 2.445030 |
| 69372463 | 3.158220 |
| 69372466 | 2.991800 |
| 69372469 | 1.997720 |
| 69372472 | 4.344520 |
| 69372473 | 3.055380 |
| 69372474 | 4.637970 |
| 69372475 | 9.499431 |
| 69372477 | 2.657180 |
| 69372480 | 1.036970 |
| 69372482 | 6.704550 |
| 69372483 | 1.218490 |
| 69372490 | 2.263090 |

In all 3 instances of valid binding sites in SMN2, a site was found (bolded). The sites exceed 5 bits. Interestingly, the 9.5 bit site is in a region, where a binding site is expected based on experimental data, but has not been localized (described as “ELEMENT 2” in the publication).

In summary, the TIA-1 model detected strong sites, but weak false positives were also present, as a result of the promiscuity of A/T rich regions being flagged. In order to eliminate false positive binding sites, the TIA1 model is preferably used in combination with a second motif for a distinct RNA binding protein, which is known to interacts with, for example, PTB. The combined motif could be computed as a R_{i,total} value, based on the strengths of each sites, and the gap surprisal distribution which relates both sites.

Although it is quite accurate, the hnRNP C model confirmed 3 of 4 published binding sites all from papers that demonstrated binding within a 20-70 nt long region, none of which described the precise location of the binding sites. The one that failed was the only one that involved a mutation which supposedly abolished an hnRNP C site, which was not detected with either of the hnRNP C models developed.

Models for both hnRNP F and hnRNP U result in high bit values for natural splice sites (both donors and acceptors). The ‘CAG’ pattern in the sequence logo is quite obvious. The possibility cannot be eliminated that the entropy minimization is biasing toward more conserved natural sites, which “contaminate” these sequences due to their proximity to the hnRNP sites. Furthermore, hnRNP F binding sites are known to have a GGG motif, which is absent from any model built from the hnRNP F data.

Hu proteins inhibit splicing by binding to intronic recognition sequences adjacent to exon 23a of NF1 (HuB, HuC, and HuD) and adjacent TIA1 sites promote recognition of the donor splice site by U1 SNRNP. See Zhu, et al. Mol Cell Biol. 2008 February; 28(4): 1240-1251. Within chr17:29,579,900-29,580,100, TIA-1 sites are present at:

| Chr. Coord. | Ri value (bits) |
|---|---|
| 29580015 | 3.791960 |
| 29580029 | 7.952610 |

A series of Hu protein binding sites has been predicted at a weak donor site in the PLOD2 gene (chromosome 3:145,795,600-145,795,750). See Yeowell, Heather N, Walker, Linda C, Mauger, David M, Seth, Puneet, Garcia-Blanco, Mariano A. TIA Nuclear Proteins Regulate the Alternate Splicing of Lysyl Hydroxylase 2, Journal of Investigative Dermatology (2009) 129, 1402-1411.

| Chr. Coord. | Ri value (in bits) |
|---|---|
| 145795604 | 6.539410 |
| 145795605 | 2.437480 |
| 145795607 | 5.573260 |
| 145795609 | 4.282010 |
| 145795610 | 3.696390 |
| 145795611 | 6.333310 |
| 145795612 | 0.722530 |
| 145795613 | 8.514270 |
| 145795614 | 6.387630 |
| 145795615 | 6.179630 |
| 145795616 | 7.204071 |
| 145795617 | 8.928380 |
| 145795618 | 0.453510 |
| 145795619 | 7.776460 |
| 145795620 | 4.122941 |
| 145795621 | 4.207820 |
| 145795622 | 9.756490 |
| 145795624 | 5.764780 |
| 145795625 | 3.915710 |
| 145795626 | 6.074350 |
| 145795627 | 0.233480 |
| 145795628 | 6.985560 |
| 145795629 | 2.751471 |
| 145795630 | 7.838311 |
| 145795631 | 8.452850 |
| 145795632 | 10.973180 |
| 145795633 | 7.993841 |
| 145795634 | 6.453230 |
| 145795635 | 7.710070 |
| 145795636 | 1.090840 |
| 145795638 | 3.965630 |
| 145795640 | 9.942340 |
| 145795641 | 8.432720 |
| 145795642 | 4.729580 |
| 145795643 | 2.373280 |
| 145795644 | 3.849880 |
| 145795645 | 5.682571 |

PTB.

Two different models were computed for PTB, which differ only by the length of the binding sites. The 6SB model is preferred based on published studies on PTB. However the 6SB model may truncate the site, which is one of the reasons why the 10SB model was also derived.

As described previously by Izquierdo et al. (2005), PTB represses inclusion of the exon 6 in FAS, which was described for TIA1 (although the PTB site is in exon 6). The interval containing the PTB binding sites span the interval chromosome 10:90,770,450-90,770,649. With the 6SB model, several potential binding sites were detected in this interval (the strongest sites are bolded).

| Chr. Coord. | Ri value (bits) |
|---|---|
| 90770505 | 1.103880 |
| 90770512 | 3.856850 |
| 90770517 | 1.824200 |
| 90770535 | 4.674070 |
| 90770543 | 4.955421 |
| 90770556 | 3.293820 |
| 90770564 | 3.055950 |
| 90770578 | 0.367950 |
| 90770582 | 3.384770 |
| 90770589 | 1.924930 |

The two strongest predicted binding sites contain the “URE6 element” described in the publication, and contain PTB “consensus” sequence, UCUU. Using the 10SB model, the corresponding sites are 2.94 and 1.13 bits, respectively, with the 3.3 bit site at 90770556 strengthening it from 3.3 to 4.5 bits.

PTB binding to the CHRNA gene has also been reported in the region, chromosome 2: 175622750-17562290 (Rahman M A, Masuda A, Ohe K, Ito M, Hutchinson D O, Mayeda A, Engel A G, Ohno K. HnRNP L and hnRNP L L antagonistically modulate PTB-mediated splicing suppression of CHRNA1 pre-mRNA. Sci Rep. 2013 Oct. 14; 3:2931.). The 7.3 bit site at position 175622764 is described in the publication (Bian Y, Masuda A, Matsuura T, Ito M, Okushin K, Engel A G, Ohno K. Tannic acid facilitates expression of the polypyrimidine tract binding protein and alleviates deleterious inclusion of CHRNA1 exon P3A due to an hnRNP H-disrupting mutation in congenital myasthenic syndrome. Hum Mol. Genet. 2009 Apr. 1; 18(7):1229-37). However, the present disclosure provides a 5.8 bit site close to the branch point.

PTB also binds to both ends of exon 9 of the gene, CAPZB (http://rnajournal.cshlp.org/content/19/5/627.long). Downstream of the exon near position 19669210, there is a 3.7 bit site situated between two ACUAA elements (with the 10 nt long ribl, 2.2 bits with the 6SB model), which are recognized by the RNA binding protein, Quaken. No other predicted sites exist in this region. Upstream of the exon around position 19669400, the published study is less precise about the location of the PTB site. The model of the instant disclosure predicted several potential sites in this region, including a 6.7 bit site ˜40 nt downstream of the exon and a 4.4 bit site ˜10 nt downstream.

HuR/ELAVL1

HuR (or ELAVL1) regulates inclusion of an exon in the FAS gene, though there is evidence to suggest it is interacting with URE6. HuR is predicted to bind at several locations across exon 6 and upstream in intron 5 (Izquierdo J M. Hu antigen R (HuR) functions as an alternative pre-mRNA splicing regulator of Fas apoptosis-promoting receptor on exon definition. J Biol. Chem. 2008 Jul. 4; 283(27):19077-84). The region upstream of the exon (chr10:90,770,450-90,770,649) has a cluster of strong HuR binding sites:

| Chr. Coord | Ri value (in bits) |
|---|---|
| 90770471 | 6.351841 |
| 90770472 | 8.330290 |
| 90770475 | 7.383730 |
| 90770477 | 5.040200 |

Within the exon, there is only a single cluster of strong binding sites, which coincides with the location of the URE6 element, as indicated in the article:

| Chr. Coord | Ri value (in bits) |
|---|---|
| 90770535 | 3.071350 |
| 90770538 | 4.882600 |
| 90770541 | 4.882600 |
| 90770542 | 2.393560 |
| 90770543 | 9.590730 |

HuR exhibits documented binding to the ATM gene. However, binding did not impact the mRNA splicing profile of this gene (http://www.ncbi.nlm.nih.gov/pubmed/21858080). There are 9 consecutive thymine residues, which results in a set of strong binding sites, corresponding to the interval described in the paper (˜80 nucleotides in length).

| Chr. Coord | Ri value (in bits) |
|---|---|
| 108141430 | 3.633660 |
| 108141431 | 7.772871 |
| 108141432 | 12.418920 |
| 108141433 | 12.418920 |
| 108141434 | 12.418920 |
| 108141435 | 2.882740 |

In Hu et al. Mol Cell Biol. 2008 February; 28(4): 1240-1251 (cited previously for TIA-1), the authors indicate that multiple Hu proteins bind to exon 23a of NF1. Our HuR model predicts a number candidate binding sites in this region.

| Chr. Coord. | Ri (in bits) |
|---|---|
| 29579831 | 2.263210 |
| 29579832 | 4.191080 |
| 29579833 | 3.633660 |
| 29579834 | 7.772871 |
| 29579835 | 2.882740 |
| 29579836 | 0.863631 |
| 29579837 | 7.102510 |

In the publication, the TIA1 site is described as adjacent to a Hu binding site downstream of the exon. 9.3 and 5.5 bit HuR binding sites were found (at pos. 29580034-35) immediately upstream and one 7.0 bit HuR site at pos. 29580047 downstream of the TIA1 site.

hnRNP A1

The following study shows that hnRNAP A1 regulates splicing of the ATM gene (Pastor T, Pagani F. Interaction of hnRNPA1/A2 and DAZAP1 with an Alu-derived intronic splicing enhancer regulates ATM aberrant splicing. PLoS One. 2011; 6(8):e23349) and binds within a 35 nucleotide interval circumscribing position 108141450.

| Chr. Coord | Ri value (in bits) |
|---|---|
| 108141439 | 5.652870 |
| 108141457 | 1.664050 |
| 108141469 | 4.653870 |

A sequence variant creates an hnRNP A1 site within ETFDH (also HNRNP A2/B1 and H). See Olsen et al. (2014).

This exonic variant at 159601742 was analyzed by information analysis to assess the predicted change in hnRNP A1 site strength. This exon itself is non-constitutive, and it is predicted that this variant increases the hnRNP A1 splicing suppressor strength, thereby increasing exon skipping (hnRNP A1 site at pos. 159601740, with R_{i,initial}=−11.16->R_{i,final}=5.94 bits).

In addition, a weak hnRNP H binding site is created (0.62 bits at pos.15961742), and another pre-existing site is strengthened (3.79->4.03 bits at pos. 15960173). An preexisting 6.9 bit site 17 nt downstream of the 4.0 bit site was also observed.

Analysis of this mutation with the hnRNP A2/B1 exon silencer model below did not detect any overlapping or novel binding sites.

In cases where a weak regulatory site overlaps a stronger site, proteins capable of binding to the weak site are likely to be displaced by the protein with the higher affinity site (stronger site). This scenario dramatically simplifies the analysis of these complex events, because when multiple binding sites are altered by a mutation, the exon definition calculation can effectively ignore the weak binding sites. Changes to total information content from effects on multiple binding sites can be reduced to fewer terms when the overlapping binding sites from different proteins have significant differences in overall binding affinity, namely, information content.

hnRNP A2B1

A different variant in another gene was found to alter strengths in splicing regulatory sequences, bound by SFSR1 and hnRNP A1, in an alternative exon of the ACADM gene (Bruun G H, Doktor T K, Andresen B S. A synonymous polymorphic variation in ACADM exon 11 affects splicing efficiency and may affect fatty acid oxidation. Mol. Genet. Metab. 2013 September-October; 110(1-2):122-8). c.1161A>G improves exon 11 inclusion in ACADM. The A form has been experimentally shown to increase hnRNP A1 binding, whereas the G allele binds SFSR1 (SF2/ASF) with higher affinity. Our predictions follow the experimental results precisely(hnRNP A1 at coordinate 76227021 is reduced in strength 6.12->1.37 bits, and SFSR1 (SF2/ASF) is increased −3.08->2.77 bits.

The gap surprisal distributions for ELAVL1-PTB-TIA1-hnRNPH are shown in FIG. 13.

### Example 6

### Failing Binding Site Models as a Result of Data Insufficiency or Bias in the Source Data

(A) Data insufficiency. Other sources of data were tested to construct information theory based models. In particular, models were derived from the SpliceAID-F database (Guiletti et al. SpliceAid-F: a database of human splicing factors and their RNA-binding sites Nucl. Acids Res. 41(D1):D125-D13). In contrast with the CLIP-Seq datasets, this database has been manually curated from published sites of 71 different RNA binding proteins. In order to ensure that the individual information contents of binding sites were distinguishable, models were developed for proteins in which >20 binding sites had been ascertained. However, PoWeMaGen disqualified a substantial number of motifs derived from this data source (because these sites had negative Ri values, and according to theory, should not be capable of binding protein), resulting in models built from 10-15 sites, which led to large confidence intervals in Rᵢ values. The elimination of some of the sites during analysis may lead to models that are based on too few sites and have questionable accuracy. After disqualifying these models, on PWM based on hnRNP D and hnRNP I remained. The hnRNP D model is a low complexity binding site that lacks specificity in long polyT-rich regions, resulting in a series of consecutive positive Rᵢ values for predicted adjacent binding sites. Interestingly, the same literature publications would frequently describe HuR binding as well at these sites, as another polyT binding protein. The hnRNP I model derived by entropy minimization-based alignment had low sensitivity, failing to detect known binding sites in about 50% of cases, and those sites it did correctly predict were usually quite weak, i.e. <3 bits.

(B) Sequence bias in the dataset. A CLIP-seq based SRSF1 model (i.e. ASF/SF2) failed to predict of the effect of a G to C substitution in a known SRSF1 binding site (Guo et al. 2013, reference follows). Although it had accurately predicted the presence of 4 sites described in 3 other publications, the particular G to C mutation which was shown to significantly decrease SRSF1 binding in a laboratory pulldown experiment, was predicted to have the opposite effect, namely, to strengthen the site. The previous SFSR1 model on ASSEDA (Mucaki et al. 2013) correctly predicted that the mutation abolished the site, but the site in the unmutated reference gene sequence was predicted to be weak (1.2 bits). This suggests that the underlying data used to create the Clip-Seq based information model are biased towards certain motifs, and do not comprehensively cover the genome-wide distribution of SRSF1 binding sites. This paper also contained a mutation which abolished an hnRNP A1 site, which was predicted correctly by the CLIP-Seq based hnRNP A1 model (5.1->−11.2 bits). See Guo R, Li Y, Ning J, Sun D, Lin L, Liu X. HnRNP A1/A2 and SF2/ASF regulate alternative splicing of interferon regulatory factor-3 and affect immunomodulatory functions in human non-small cell lung cancer cells. PLoS One. 2013 Apr. 29; 8(4):e62729.

### Example 7

### Application of R_{i,total} to Splicing Regulation—Experimental Validation of to BRCA1 and BRCA2 Gene Mutations Predicted by Exon Definition Analysis

Numerous unclassified variants (UVs) have been identified in splicing regions of disease-associated genes and their characterization as pathogenic mutations or benign polymorphisms is crucial for the understanding of their role in disease development. The number of these alterations has increased considerably as a consequence of next generation sequencing analyses and confounds distinction of disease variants.

The aim of the present study was to assess the splice isoforms predicted by ASSEDA, through qPCR-based analyses. Where mRNA was available, we compared cryptic isoforms computed by exon definition analysis and their predicted abundance to results from semi quantitative RT-PCR and quantitative RT-PCR studies. Twenty-four UVs in BRCA genes were previously characterized by conventional end-point Reverse Transcriptase-PCR (RT-PCR) [1]. Nineteen splicing mutations and 5 non-spliceogenic base changes were observed. All variants were re-evaluated using ASSEDA (http://ossify.sg.csd.uwo.ca), and the predicted isoforms were annotated (Table 2). The value of the Window Range (i.e., the region before and after the base where the mutation takes place and where the information content of sites is calculated) was set to 450 nt.

TABLE 2
Summary of ASSEDA results and their consistency with in vitro results.

| | | | | ASSEDA isoform prediction | | |
|---|---|---|---|---|---|---|
| | Variant | | | Position | | |
| | (HGVS- | | mRNA change observed by in vitro | relative | Initial | Final |
| Gene | [symbol] ) | [symbol] | analyses [1] | to Natural Site | R total | R total |
| BRCA1 | c.547 + 2T > A | D | skipping of exon 8 | 0 | 7.8 | −10.7 |
| inactivating | | | | 138 | 7.1 | 7.1 |
| | c.4867 − 1G > A | A | skipping of exon 17 | 0 | 8.1 | −2.8 |
| | | | | −187 | 17.4 | 17.4 |
| | | | | −188 | 8.3 | 8.3 |
| | c.5322 + 1G > A | D | skipping of exon 21 | 0 | 23.3 | 4.7 |
| | | | | 215 | 13.3 | 15.3 |
| | | | | 305 | 12.8 | 12.8 |
| | c.134 + 3_134 + 6 | D | up-regulation of exon 3 [symbol] | 0 | 10.8 | 2.5 |
| | delAAGT | | | 103 | 8.2 | 8.2 |
| | c.4454G > T | D | skipping of exon 14 | 0 | 15.3 | 18.8 |
| Cryptic | c.212G > A | D | up-regulation of [symbol] exon 5q isoform | 0 | 15.2 | 12.2 |
| | | | | −22 | 14.1 | 14.1 |
| | c.212-11T> G | A | [symbol] of 59 bp [symbol] at the [symbol] isoform 5 | 0 | 8.4 | 8.8 |
| | | | | −59 | 13.8 | 1.8 |
| | | | | −47 | 11.1 | 11.1 |
| | c.441 + 2T > G | D | skipping of 62 bp at the 8′-end of exon7 | 0 | 13.5 | −51 |
| | | | | −62 | 15.2 | 15.2 |
| | | | | 275 | 10.4 | 10.4 |
| | | | | 282 | 9.7 | 9.7 |
| | c.4305 + 1G > T | D | [symbol] of 65 bp at the 5′-end intron 88 | 0 | 8.4 | −10.3 |
| | | | | −95 | 10.5 | 10.5 |
| | | | | −93 | 10.3 | 10.3 |
| | | | | 65 | 8.4 | 8.4 |
| | c.4385 + 5G > A | D | [symbol] of 65 bp at the 5′-end intron [symbol] | 0 | 8.4 | 5.2 |
| | | | | −95 | 10.5 | 10.5 |
| | | | | −93 | 10.3 | 10.3 |
| | | | | 65 | 8.4 | 8.4 |
| | c.5275 − 2del | A | skipping of exon 21; | 0 | 23.3 | 4.4 |
| | | | skipping of 8 bp at the 5′-end of exon 21 | 6 | 17.2 | 15.8 |
| | | | | [symbol] | 12.8 | 12.8 |
| | | | | 34 | 12 | 12 |
| Not | c.548 − 3delT | A | None | 0 | 7.4 | 8.2 |
| [symbol] | c.534 − 4A > G | A | none | 0 | 1.9 | 11.7 |
| | c.4097G > A | A | none | 0 | 15 | 14 |
| | c.5332A > G | A | none | 0 | 10 | 11 |
| BRCA2 | c.475 + 1G > A | D | skipping of exon 5 | 0 | 11.7 | −7 |
| inactivating | | | | 44 | 5.5 | 5.5 |
| | | | | 5 | 4.7 | 4.7 |
| | c.921G > A | D | skipping of exon 7 | 0 | 17.4 | 14.4 |
| | | | | 20 | 14.2 | 14.2 |
| | c.5117G > A | D | skipping of exon 23 | 0 | 8.1 | 6.1 |
| | | | | −85 | 12.1 | 12.1 |
| | | | | 18 | 7.8 | 7.8 |
| | | | | 48 | 8.2 | 6.2 |
| | | | | 89 | 5 | 5 |
| Leaky | c.478 − 2A >G | A | skipping of exon8, | 0 | 17.8 | 3.1 |
| | | | up-regulatin of [symbol] exon 6-8 isoform | 38 | 14.7 | 14.7 |
| | | | | 51 | 10.8 | 10.8 |
| | c.5753 − 1G > A | A | skipping of exon 72; | 0 | 11.4 | 8.5 |
| | | | skipping of exon 22 + 51 bp at the 3′-end | −71 | 12.7 | 12.7 |
| | | | of exon 23 | 382 | 11.9 | 11.9 |
| | | | | −17 | 8.8 | 8.8 |
| | | | | −63 | 8 | 8 |
| Cryptic | c.7008 + 2A > T | A | Skipping of exon 14; | 0 | 4.9 | −2.3 |
| | | | skipping of 10 bp at 5′-end of exon 14 | 248 | 4.1 | 4.1 |
| | | | skipping of 248 bp at 6′-end of exon 14 | | | |
| | c.8754 + 3G > C | D | [symbol] of 46 bp at the 5′-end of intron 21 | 0 | 18.5 | 14.9 |
| | | | | 46 | 18.3 | 18.2 |
| | | | | 8 | 14.2 | 14.2 |
| | c.7564 + [symbol] _8655 | A | skipping of 61 bp at the 6′-end of exon 23 | 0 | 8.1 | −8.4 |
| | delTTinsAA | | skipping exon 23 | 61 | 7.9 | 7.9 |
| Not | c.9118C > T | D | none | 0 | 8.1 | 8.6 |
| [symbol] | | | | | | |

| | Variant | | |
|---|---|---|---|
| | (HGVS- | | Comparison with in |
| Gene | [symbol] ) | Interpretation of ASSEDA prediction [symbol] | vitro results [symbol] |
| BRCA1 | c.547 + 2T > A | inactivating mutation; | Concordant |
| inactivating | | [symbol] 33 bp downstream | |
| | c.4867 − 1G > A | inactivating mutation; | Concordant |
| | | cryptic acceptor 187 bp upstream; | |
| | | cryptic [symbol] 193 bp upstream | |
| | c.5322 + 1G > A | inactivating mutation; | Concordant |
| | | [symbol] 216 bp downstream; | |
| | | [symbol] 306 bp downstream | |
| | c.134 + 3_134 + 6 | inactivating mutation; | Concordant |
| | delAAGT | [symbol] 108 bp downstream | |
| | c.4454G > T | Leaky mutation | Consistent |
| Cryptic | c.212G > A | Leaky mutation; | Concordant |
| | | [symbol] 22 bp upstream | |
| | c.212-11T> G | inactivating mutation; | Concordant |
| | | [symbol] 38 bp upstream; | |
| | | [symbol] 47 bp upstream | |
| | c.441 + 2T > G | inactivating mutation; | Concordant |
| | | a [symbol] 62 bp upstream; | |
| | | a [symbol] 275 bp downstream; | |
| | | a [symbol] 233 bp downstream | |
| | c.4305 + 1G > T | inactivating mutation; | Concordant |
| | | a [symbol] 95 bp upstream; | |
| | | a [symbol] 93 bp upstream; | |
| | | a [symbol] 85 bp downstream | |
| | c.4385 + 5G > A | inactivating mutation; | Concordant |
| | | a [symbol] 56 bp upstream; | |
| | | a [symbol] 93 bp upstream; | |
| | | a [symbol] 85 bp downstream | |
| | c.5275 − 2del | inactivating mutation; | Concordant |
| | | a [symbol] isororm nad [symbol] ; | |
| | | a [symbol] 55 bp upstream; | |
| | | a [symbol] 94 bp downstream | |
| Not | c.548 − 3delT | Negligible change in R total_polymorphism | Concordant |
| [symbol] | c.534 − 4A > G | Negligible change in R total_polymorphism | Concordant |
| | c.4097G > A | Negligible change in R total_polymorphism | Concordant |
| | c.5332A > G | [symbol] site_polymorphism | Concordant |
| BRCA2 | c.475 + 1G > A | inactivating mutation; | Concordant |
| inactivating | | a [symbol] 44 bp downstream; | |
| | | a [symbol] 5 bp upstream | |
| | c.921G > A | [symbol] mutation; | Concordant |
| | | [symbol] 70 bp upstream | |
| | c.5117G > A | [symbol] mutation; | Consistent |
| | | a [symbol] 86 bp upstream; | |
| | | a [symbol] 18 bp downstream; | |
| | | a [symbol] 43 bp downstream; | |
| | | a [symbol] 89 bp downstream | |
| Leaky | c.478 − 2A >G | inactivating mutation; | Concordant |
| | | [symbol] 68 bp upstream; | |
| | | [symbol] 61 bp upstream | |
| | c.5753 − 1G > A | inactivating mutation; | Concordant for |
| | | [symbol] 71 bp upstream; | inactivation; |
| | | [symbol] 382 bp downstream | discordant for crypto |
| | | [symbol] 17 bp upstream | isoform |
| | | [symbol] 63 bp upstream | |
| Cryptic | c.7008 + 2A > T | inactivating mutation; | Concordant for |
| | | [symbol] 248 bp downstream | inactivation and one of |
| | | | two crypticisoform |
| | c.8754 + 3G > C | [symbol] mutation; | Concordant |
| | | [symbol] 48 bp downstream; | |
| | | [symbol] 8 bp downstream | |
| | c.7564 + [symbol] _8655 | inactivating mutation; | Concordant |
| | delTTinsAA | [symbol] 51 bp downstream | |
| Not | c.9118C > T | [symbol] site_polymorphism | Concordant |
| [symbol] | | | |

^{a}SS: splice site.
^{b}The predicted isoforms virified by qPCR analyses are indicated in bold, detected isofomrs (green), not detected isoforms (red).
^{c}Concordant: experimentally virified isoforms are predicted. Consistent: reduced exon definition predicting leaky splicing is in agreement with exon skipping, but residual expression of full-length transcript from mutated allele not detected. [symbol]
indicates data missing or illegible when filed

The qPCR assays were performed using the KAPA SYBR FAST Universal qPCR kit (KAPA BIOSYSTEMS) and examined on an Eco Real-Time PCR System (Illumina). The level of expression of each isoform was measured relative to the level of expression of the same isoform in a reference sample. In addition, the level of expression of each isoform considered in the assay was normalized to the expression of CCDC137, as a reference gene. For each assay, uniform length amplicons were generated from reverse transcripts using isoform-specific splice junction primers. For the BRCA1 c. 4987-1G>A the normal transcript, the Δexon17 isoform and the transcript derived from the partial retention of intron 16 (187 bp at the 3′-end) were analyzed. For the BRCA1 c.5278-2delA the normal transcript, the Δexon21 isoform and the transcripts derived from the partial skipping of exon 21 (8 bp at the 5′-end) and the partial retention of intron 20 (51 bp at the 3′-end) were verified. In both analyses, a fragment spanning BRCA1 exon 8-9 junction was generated to serve as an internal reference.

ASSEDA detected all splicing mutations (n=19) and 9 of 11 cryptic isoforms observed in UV carriers (Table 1). Non-spliceogenic variants (n=5) did not exhibit significant changes in exon information. Cryptic isoforms of lower abundance not seen in previous analyses were also predicted (between 0 and 4 transcripts per mutation). Verification of these predictions by qPCR is currently ongoing. At present, the BRCA1 c. 4987-1G>A and c.5278-2delA mutations were analyzed. The full-length and the Δexon17 isoforms for the BRCA1 c. 4987-1G>A mutation and the full-length, the Δexon21 and the Δexon21q isoforms for the 5278-2delA were confirmed. However, additional low abundance isoforms predicted by ASSEDA were not observed in qPCR experiments, as expected.

Based on these results, it is conclude that information theory-based exon definition comprehensively detects the experimentally-verified repertoire of mutant isoforms by end point RT-PCR in carriers of the investigated UVs. Preliminary results show that qPCR analyses can determine which of the many potential intronic cryptic splice sites that are predicted by ASSEDA are potentially relevant and which ones can be dismissed as being irrelevant to pathogenicity.

The loss of exon identity due to the combined activation of binding sites associated with silencing of exon recognition and loss of binding sites recognized by exon enhancers has been shown. See Sterne-Weiler T, Howard J, Mort M, Cooper D N, Sanford J R, Loss of exon identity is a common mechanism of human inherited disease. Genome Res. 2011 October; 21(10):1563-71. However, although Sterne-Weiler et al. implicated specific hexamer sequences as contributing to exon skipping, and the splicing factors PTB and SRp20 in regulation of exon skipping, the context of these sequences with respect to their distance to the adjacent constitutive splice sites was not addressed or considered.

U.S. Pat. No. 8,361,979 B2 describes a method for inducing exon skipping by targeting oligonucleotide sequences to Serine-Arginine rich proteins that promote exon inclusion. However, the method of the '979 patent does not recognize the role that hnRNP A1 plays in proofreading of exon boundaries, nor does it consider that the proximity between this splicing regulatory sequence and the adjacent constitutive splice site is important for exon definition (i.e. Targeting neighboring and distant binding sites is likely to have different effects), and does not transform that distance into units of bits, i.e. Gap surprisal, so as to compute R_{i,total}, the method described in the instant invention for predicting exons that are recognized and processed in unspliced heteronuclear RNAs.

### Example 8

### Exon Definition Analysis Reveals a Previously Unrecognized, but Common Mechanism of Exon Skipping Based on hnRNP A1 Cryptic Site Generation

Recursive stop-gain mutation c.5791C>T (rs144567652) in FANCM abolishes exon definition, inducing exon skipping and is a risk factor for familial breast cancer. The c.5791C>T mutation originates a stop codon at residue 1931 generating the loss of 118 amino-acids from the FANCM C-terminus that destroys the functional domain that mediates the interaction with FAAP24 (Ciccia et al. 2007) and DNA translocation (Rosado et al. 2009). However, functional analyses in lymphoblastoid cell lines obtained from two mutation carriers resulted a very low level of the mutated mRNA, suggesting that the c.5791C>T has a loss of function effect. This result was unexpected because this mutation occurs in the penultimate exon of the gene, where nonsense mediated decay, the predominant cellular mechanism of mRNA surveillance of premature stop codons, is not expected to cause significant mRNA degradation due to its close proximity to the 3′ untranslated region of the mRNA (Shoemaker E and Green R, Nature Struct. & Mol. Biol. 19: 594-601, 2012).

Information theory-based mutation analysis was used to assess the impact of the variant on splicing regulatory binding sites that regulate definition of the exon. The mutation is predicted to create an overlapping 4.6 bit hnRNP A1 binding site (c.5790_{—}5795; Mucaki et al. 2013), which completely suppresses normal exon recognition (R_{i,total}: 3.4 (C)->−2.6 (U) bits, inactivating exon recognition and results in complete exon skipping. The novel hnRNP A1 binding site sequence is frequently present in sites crosslinked to hnRNP A1 protein (Huelga et al. 2012). The frequencies of the normal and mutated FANCM hnRNPA1 sites from the sequences that were used to build the model for the present disclosure shows 140431 binding sites total in the model. The wild type site (CCGAAU) was not present, which is consistent with its negative Ri value. However, the mutant site CUGAAU was present 716 times in set of binding sites crosslinked to the protein. These are experimental data from crosslinking experiments using an antibody against hnRNP A1 to pull down these sequences. The reason why exon skipping occurs is related to one of the key functions of hnRNP A1. HnRNP A1 proofreads U2AF binding at the 3′ splice site. It also directly interacts with the 5′ splice site. See N. R. Zearfoss, E S. Johnson and S P. Ryder, hnRNP A1 and secondary structure coordinate alternative splicing of Mag, RNA (2013) 19: 948-957. For this protein binding site (Tavenez et al. 2012), exonic hnRNP A1 sites distant from known splice sites are very rare in the transcriptome (FIG. 2, which is consistent with abrogration of exon definition and exon skipping (Olsen et al. 2014). Skipping of exon 22 prematurely terminates translation after incorporating 11 frameshifted residues from exon 23, and the loss of 143 amino-acids from the FANCM C-terminus (p.Gly1906Alafs11*). This recursive property which introduces a premature stop codon further upstream of p.R1931X ensures that the mutant FANCM is incapable of complexing with FAAP24 or binding DNA.

The opal codon in FANCM contained the core sequence of the novel hnRNP A1 site (positions 1-3 of FIG. 14) in FANCM and the amber codon also contains conserved nucleotides in this binding site (positions 0-2 of FIG. 14). It appears that creation at hnRNP A1 coincident stop codons is a general mechanism to ensure exon skipping at these sites. Because the Ri(b,l) weight matrix that other CGA>TGA (Arg>Ter) mutations would be expected to activate hnRNP A1 sites, the National Center for Biotechnology Information's ClinVar database was searched with search term: (“stop gain”[Molecular consequence]) and all of the Arg>Ter mutations were analyzed with the instant invention. Arg>Ter is a very common stop-gain mutation in this database, which consists of published mutations as well as those contributed by clinical molecular diagnostic laboratories. More than 80% of the mutations analyzed create an hnRNP A1 site exceeding 3.5 bits in strength (in some cases, creating 2 sites). If the site is more than 40 nucleotides distant from the adjacent splice site, the reduction in Ri,total is quite significant and the difference in R_{i,total} values of the normal and mutant exon exceeds 3 bits (8 fold abundance), supporting a high level of exon skipping. We noted that instant invention presents potential cryptic isoforms with R_{i,total} values exceeding that of the mutated exon. Because the hnRNP A1 mutation affects acceptor site recognition, it is unlikely that these isoforms will be present, especially in instances where the cryptic splice site is a donor, and the natural acceptor is shared between the constitutive and cryptic isoforms.

Even assuming that triplet periodicity of exon lengths is random, one-third of all exon skipping events would not alter the reading frame. Nonsense mutations are generally acknowledged as pathogenic, are frequently lethal, and certainly reduce fecundity. It is well known in the art that non-sense codons induce exon skipping, as an alternative to nonsense mediated decay (T. Casci, Molecular evolution: Dealing with nonsense, Nature Reviews Genetics 12, 805). However, the specific mechanisms by which this phenomenon occurs have only been the subject of speculation, with limited specific evidence or mechanism as proven explanations for the phenomenon. Natural selection has evolved this mechanism to skip this abundant nonsense codon, TGA. For those exon skipping events that preserve the reading frame, the skipping event may result in less severe phenotypes, depending on how the structure of the protein is deformed by the loss of a stretch of amino acids. The periodic behavior of the gap surprisal function for exon lengths that are multiples of three nucleotides, suggests selection favoring exons of length that preserve the open reading frame.

Individual splicing mutations identified by exon definition may be validated by RT-PCR or qRT-PCR.

Changes may be made in the above methods without departing from the scope hereof. It should be noted that the matter contained in the above description or shown in the accompanying drawings should be interpreted as illustrative and not in a limiting sense. The following claims are intended to cover generic and specific features described herein, as well as statements of the scope of the present methodology, which, as a matter of language, might be said to fall therebetween.

It should be understood that suitable equivalents may be used in place of or in addition to the various instruments, components or compositions, the function and use of such substitute or additional components being held to be familiar to those skilled in the art and are therefore regarded as falling within the scope of the present disclosure. Therefore, the present examples are to be considered as illustrative and not restrictive, and the present disclosure is not to be limited to the details given herein but may be modified within the scope of the appended claims.

### REFERENCES

The following references are either cited in this disclosure or are of relevance to the present disclosure. All documents listed below, along with other papers, patents and publication of patent applications cited throughout this disclosures, are hereby incorporated by reference as if the full contents are reproduced herein.
Barash, Y., Calarco, J. A., Gao, W., Pan, Q., Wang, X., Shai, O., Blencowe, B. J., Frey, B. J. 2010. Deciphering the splicing code. Nature 465(7294): 53-9, 2010.
Berget S M. 1995. Exon recognition in vertebrate splicing. J Biol. Chem. 270:2411-2414.
Bolisetty M T, Beemon K L. 2012. Splicing of internal large exons is defined by novel cis-acting sequence elements. Nucleic Acids Res. 40(18):9244-54.
Cartegni L., Krainer A. R. 2002. Disruption of an SF2/ASF-dependent exonic splicing enhancer in SMN2 causes spinal muscular atrophy in the absence of SMN1. Nat. Genet. 30:377-384.
Churbanov A, Igor B. Rogozin, Jitender S. Deogun and Hesham Ali, Method of predicting Splice Sites based on signal interactions, Biology Direct 1 (2006), no. 10.
Churbanov A, Igor Vorechovsky and Chindo Hicks A method of predicting changes in human gene splicing induced by genetic variants in context of cis-acting elements, BMC Bioinformatics 2010, 11:22
Claes K, Vandesompele J, Poppe B, Dahan K, Coene I, De Paepe A, Messiaen L. 2002. Pathological splice mutations outside the invariant AG/GT splice sites of BRCA1 exon 5 increase alternative transcript levels in the 5′ end of the BRCA1 gene. Oncogene. 21:4171-4175.
Claes K, Poppe B, Machackova E, Coene I, Foretova L, De Paepe A, and Messiaen L. 2003. Differentiating pathogenic mutations from polymorphic alterations in the splice sites of BRCA1 and BRCA2. Genes Chromosomes Cancer. 37:314-320.
Clark F, Thanaraj T A. 2002. Categorization and characterization of transcript-confirmed constitutively and alternatively spliced introns and exons from human. Hum Mol. Genet. 11: 451-464.
Clayero S, Pérez B, Rincón A, Ugarte M, Desviat L R. 2004. Qualitative and quantitative analysis of the effect of splicing mutations in propionic acidemia underlying non-severe phenotypes. Hum Genet. 115(3):239-47.
Cook K B, Kazan H, Zuberi K, Morris Q, and Hughes T R. 2011. RBPDB: a database of RNA-binding specificities. Nucleic Acids Res. 39:D301-8.
Cover T M, Thomas J A. 2006. Elements of information theory. Wiley-Interscience, Hoboken, N.J.: p. 748.
Dalgleish R, Flicek P, Cunningham F, Astashyn A, Tully R E, Proctor G, Chen Y, McLaren W M, Larsson P, Vaughan B W, Beroud C, Dobson G et al. 2010. Locus Reference Genomic sequences: an improved basis for describing human DNA variants. Genome Med. 2:24.
De Conti L, Baralle M, Buratti E. 2012. Exon and intron definition in pre-mRNA splicing. Wiley Interdiscip Rev RNA. doi: 10.1002/wrna.1140.
Divina P, Kvitkovicova A, Buratti E, Vorechovsky I. 2009. Ab initio prediction of mutation-induced cryptic splice-site activation and exon skipping. Eur J Hum Genet. 17:759-765.
Dominski Z, Kole R. 1991. Selection of splice sites in pre-mRNAs with short internal exons. Mol Cell Biol. 11(12):6075-83.
Dominski Z, Kole R. 1992. Cooperation of pre-mRNA sequence elements in splice site selection. Mol Cell Biol. 12:2108-2114.
Goina E, Skoko N, Pagani F. 2008. Binding of DAZAP1 and hnRNPA1/A2 to an exonic splicing silencer in a natural BRCA1 exon 18 mutant. Mol Cell Biol. 28(11):3850-60.
Graveley B R, Maniatis T. 1998. Arginine/serine-rich domains of SR proteins can function as activators of pre-mRNA splicing. Mol. Cell. 1:765-771.
Goren A, Kim E, Amit M, Vaknin K, Kfir N, Ram O, Ast G. 2010. Overlapping splicing regulatory motifs—combinatorial effects on splicing. Nucleic Acids Res. 38:3318-3327.
Hwang D Y, Cohen J B. 1997. U1 small nuclear RNA-promoted exon selection requires a minimal distance between the position of U1 binding and the 3′ splice site across the exon. Mol Cell Biol. 17:7099-7107.
Ibrahim E C, Schaal T D, Hertel K J, Reed R, Maniatis T. 2005. Serine/arginine-rich protein-dependent suppression of exon skipping by exonic splicing enhancers. Proc Natl Acad Sci USA. 102:5002-5007.
Jaynes E. Information Theory and Statistical Mechanics. Phys. Rev. 106, 620-630 (1957).
Lim K H, Ferraris L, Filloux M E, Raphael B J, Fairbrother W G. 2011. Using positional distribution to identify splicing elements and predict pre-mRNA processing defects in human genes. Proc Natl Acad Sci USA. 108(27):11093-8.
Liu H X, Zhang M, Krainer A R. 1998. Identification of functional exonic splicing enhancer motifs recognized by individual SR proteins. Genes Dev. 12:1998-2012.
Liu H X, Chew S L, Cartegni L, Zhang M Q, Krainer A R. 2000. Exonic splicing enhancer motif recognized by human SC35 under splicing conditions. Mol. Cell. Biol. 20:1063-1071.
Macias-Vidal J, Rodes M, Hernandez-Perez J M, Vilaseca M A, Coll M J. 2009. Analysis of the CTNS gene in 32 cystinosis patients from Spain. Clin Genet. 76:486-489.
Mucaki E J, Ainsworth P, Rogan P K. 2011. Comprehensive prediction of mRNA splicing effects of BRCA1 and BRCA2 variants. Hum Mutat. 32:735-42.
Mucaki E J, Shirley B C, Rogan P K. 2013. Prediction of Mutant mRNA Splice Isoforms by Information Theory-Based Exon Definition. Hum Mutat. 34:557-65.
Nalla V K, Rogan P K. 2005. Automated splicing mutation analysis by information theory. Hum Mutat. 25:334-342.
Olsen et al., The ETFDH c.158A>G Variation Disrupts the Balanced Interplay of ESE- and ESS-Binding Proteins thereby Causing Missplicing and Multiple Acyl-CoA

Dehydrogenation Deficiency. Human Mutation, Volume 35, Issue 1, pages 86-95 (2014).
Robberson B L, Cote G J, and Berget S M. 1990. Exon definition may facilitate splice site selection in RNAs with multiple exons. Mol Cell Biol. 10:84-94.
Rogan P K, Faux B M, Schneider T D. 1998. Information analysis of human splice site mutations. Hum Mutat. 12:153-171.
Rogan P K, Svojanovsky S R, Leeder J S. 2003. Information theory-based analysis of CYP219, CYP2D6 and CYP3A5 splicing mutations. Pharmacogenetics. 13:207-18.
Rogan P K. 2009. Ab Initio Exon Definition Using an Information Theory-based Approach. Biochemistry Publications. Paper 10. http://ir.lib.uwo.ca/biochempub/10.
Rutter J L, Goldstein A M, Davila M R, Tucker M A, Struewing J P. 2003. CDKN2A point mutations D153spl(c.457G>T) and IVS2+1G>T result in aberrant splice products affecting both p16INK4a and p14ARF. Oncogene. 22:4444-8.
Sanz D J, Acedo A, Infante M, Duran M, Perez-Cabornero L, Esteban-Cardenosa E, Lastra E, Pagani F, Miner C, Velasco E A. 2010. A high proportion of DNA variants of BRCA1 and BRCA2 is associated with aberrant splicing in breast/ovarian cancer patients. Clin Cancer Res. 16:1957-67.
Schneider T D, Stormo G D, Yarus M A, Gold L. 1984. Delila system tools. Nucleic Acids Res. 12:129-140.
Schneider T D. 1997. Information content of individual genetic sequences. J Theor Biol. 189:427-441.
Shultzaberger R K, Bucheimer R E, Rudd K E, Schneider T D. 2001. Anatomy of Escherichia coli ribosome binding sites. J Mol. Biol. 313:215-228.
Smith P J, Zhang C, Wang J, Chew S L, Zhang M Q, Krainer A R. 2006. An increased specificity score matrix for the prediction of SF2/ASF-specific exonic splicing enhancers. Hum Mol. Genet. 15(16):2490-508.
Spurdle A B, Healey S, Devereau A, Hogervorst F B, Monteiro A N, Nathanson K L, et al. ENIGMA—evidence-based network for the interpretation of germline mutant alleles: an international initiative to evaluate risk and clinical significance associated with sequence variation in BRCA1 and BRCA2 genes. Hum Mutat. 2012; 33(1):2-7.
Stamm S, Riethoven J J, Le Texier V, Gopalakrishnan C, Kumanduri V, Tang Y, Barbosa-Morais N L, Thanaraj T A. 2006. ASD: a bioinformatics resource on alternative splicing. Nucl Acids Res. 34(suppl 1):D46-55.
Thomassen M, Ana Blanco, Marco Montagna, Thomas V. O. Hansen, Inge S. Pedersen, Sara Gutierrez-Enriquez, Mireia Menendez, Laura Fachal, Marta Santamarina, Ane Y. Steffensen, Lars Jonson, Simona Agata, Phillip Whitey, Silvia Tognazzo, Eva Tornero, Uffe B. Jensen, Judith Balmana, Torben A. Kruse, David E. Goldgar, Conxi Lazaro, Orland Diez, Amanda B. Spurdle, Ana Vega, Characterization of BRCA1 and BRCA2 splicing variants: a collaborative report by ENIGMA consortium members Breast Cancer Res Treat. 2012 April; 132(3):1 009-23
Tompson S W, Ruiz-Perez V L, Blair H J, Barton S, Navarro V, Robson J L, Wright M J, Goodship J A. 2007. Sequencing EVC and EVC2 identifies mutations in two-thirds of Ellis-van Creveld syndrome patients. Hum Genet. 120:663-670.
Tribus M. 1961. Thermostatics and thermodynamics: an introduction to energy, information and states of matter, with engineering applications. Van Nostrand, Princeton, N.J.: p. 649.

### REFERENCES FOR MUTATIONS IN FIG. 8 ARE LISTED BELOW

¹ Santisteban I, Arredondo-Vega F X, Kelly S, Mary A, Fischer A, Hummell D S, Lawton A, Sorensen R U, Stiehm E R, Uribe L. 1993. Novel splicing, missense, and deletion mutations in seven adenosine deaminase-deficient patients with late/delayed onset of combined immunodeficiency disease. Contribution of genotype to phenotype. J Clin Invest 92:2291-2302.
² Sanz D J, Acedo A, Infante M, Duran M, Perez-Cabornero L, Esteban-Cardenosa E, Lastra E, Pagani F, Miner C, Velasco E A. 2010. A high proportion of DNA variants of BRCA1 and BRCA2 is associated with aberrant splicing in breast/ovarian cancer patients. Clin Cancer Res 16:1957-67.
³ Chen X, Truong T T, Weaver J, Bove B A, Cattie K, Armstrong B A, Daly M B, Godwin A K. 2006. Intronic alterations in BRCA1 and BRCA2: effect on mRNA splicing fidelity and expression. Hum Mutat 27:427-435.
⁴ Claes K, Vandesompele J, Poppe B, Dahan K, Coene I, De Paepe A, Messiaen L. 2002. Pathological splice mutations outside the invariant AG/GT splice sites of BRCA1 exon 5 increase alternative transcript levels in the 5′ end of the BRCA1 gene. Oncogene 21:4171-4175.
⁵ Claes K, Poppe B, Machackova E, Coene I, Foretova L, De Paepe A, and Messiaen L. 2003. Differentiating pathogenic mutations from polymorphic alterations in the splice sites of BRCA1 and BRCA2. Genes Chromosomes Cancer 37:314-320.
⁶ Caux-Moncoutier V, Pages-Berhouet S, Michaux D, Asselain B, Castera L, De Pauw A, Buecher B, Gauthier-Villars M, Stoppa-Lyonnet D, Houdayer C. 2009. Impact of BRCA1 and BRCA2 variants on splicing: clues from an allelic imbalance study. Eur J Hum Genet. 17:1471-1480.
⁷ Gutierrez-Enriquez S, Coderch V, Masas M, Balmana J, Diez O. 2009. The variants BRCA1 IVS6-1G>A and BRCA2 IVS15+1G>A lead to aberrant splicing of the transcripts. Breast Cancer Res Treat 117:461-465.
⁸ Campos B, Diez O, Domenech M, Baena M, Balmana J, Sanz J, Ramirez A, Alonso C, Baiget M. 2003. RNA analysis of eight BRCA1 and BRCA2 unclassified variants identified in breast/ovarian cancer families from Spain. Hum Mutat 22:337.
⁹ Rutter J L, Goldstein A M, Davila M R, Tucker M A, Struewing J P. 2003. CDKN2A point mutations D153spl (c.457G>T) and IVS2+1G>T result in aberrant splice products affecting both p16INK4a and p14ARF. Oncogene 22:4444-8.
¹⁰ Harland M, Mistry S, Bishop D T, Bishop January 2001. A deep intronic mutation in CDKN2A is associated with disease in a subset of melanoma pedigrees. Hum Mol Genet. 23:2679-2686.
¹¹ Macias-Vidal J, Rodes M, Hernandez-Perez J M, Vilaseca M A, Coll M J. 2009. Analysis of the CTNS gene in 32 cystinosis patients from Spain. Clin Genet. 76:486-489.
¹² Tompson S W, Ruiz-Perez V L, Blair H J, Barton S, Navarro V, Robson J L, Wright M J, Goodship J A. 2007. Sequencing EVC and EVC2 identifies mutations in two-thirds of Ellis-van Creveld syndrome patients. Hum Genet. 120:663-670.
¹³ Arranz J A, Pinol F, Kozak L, Perez-Cerda C, Cormand B, Ugarte M, Riudor E. 2002. Splicing mutations, mainly IVS6-1 (G>T), account for 70% of fumarylacetoacetate hydrolase (FAH) gene alterations, including 7 novel mutations, in a survey of 29 tyrosinemia type I patients. Hum Mutat 20:180-188.
¹⁴ Schloesser M, Hofferbert S, Bartz U, Lutze G, Lammle B, Engel W. 1995. The novel acceptor splice site mutation 11396(G->A) in the factor XII gene causes a truncated transcript in cross-reacting material negative patients. Hum Mol Genet. 4:1235-1237.
¹⁵ Lapoumeroulie C, Acuto S, Rouabhi F, Labie D, Krishnamoorthy R, Bank A. 1987. Expression of a beta thalassemia gene with abnormal splicing. Nucleic Acids Res 15:8195-8204.
¹⁶ Treisman R, Orkin S H, Maniatis T. 1983. Specific transcription and RNA splicing defects in five cloned beta-thalassaemia genes. Nature 302: 591-596.
¹⁷ Vidaud M, Gattoni R, Stevenin J, Vidaud D, Amselem S, Chibani J, Rosa J, Goossens M. 1989. A 5′ splice-region G-C mutation in exon 1 of the human beta-globin gene inhibits pre-mRNA splicing: a mechanism for beta+-thalassemia. Proc Natl Acad Sci USA 86:1041-1045.
¹⁸ Atweh G F, Anagnou N P, Shearin J, Forget B G, Kaufman R E. 1985. Beta-thalassemia resulting from a single nucleotide substitution in an acceptor splice site. Nucleic Acids Res 13:777-790.
¹⁹ Bunge S, Steglich C, Zuther C, Beck M, Morris C P, Schwinger E, Schinzel A, Hopwood J J, Gal A. 1993. Iduronate-2-sulfatase gene mutations in 16 patients with mucopolysaccharidosis type II (Hunter syndrome). Hum Mol Genet. 2:1871-1875.
²⁰ Erdmann J, Raible J, Maki-Abadi J, Hummel M, Hammann J, Wollnik B, Frantz E, Fleck E, Hetzer R, Regitz-Zagrosek V. 2001. Spectrum of clinical phenotypes and gene variants in cardiac myosin-binding protein C mutation carriers with hypertrophic cardiomyopathy. J Am Coll Cardiol 38:322-330.
²¹ Dworniczak B, Aulehla-Scholz C, Kalaydjieva L, Bartholome K, Grudda K, Horst J. 1991. Aberrant splicing of phenylalanine hydroxylase mRNA: the major cause for phenylketonuria in parts of southern Europe. Genomics 11:242-246.
²² Maciolek N L, Alward W L, Murray J C, Semina E V, McNally M T. 2006. Analysis of RNA splicing defects in PITX2 mutants supports a gene dosage model of Axenfeld-Rieger syndrome. BMC Med Genet. 7:59.
²³ Vega Al, Pérez-Cerdá C, Desviat L R, Matthijs G, Ugarte M, Pérez B. 2009. Functional analysis of three splicing mutations identified in the PMM2 gene: toward a new therapy for congenital disorder of glycosylation type Ia. Hum Mutat 30:795-803.

### REFERENCES FOR MUTATIONS IN FIG. 9 ARE LISTED BELOW

¹ Miyajima H, Miyaso H, Okumura M, Kurisu J, Imaizumi K. 2002. Identification of a cis-acting element for the regulation of SMN exon 7 splicing. J Biol. Chem. 277(26):23271-7.
² Heintz C, Dobrowolski S F, Andersen H S, Demirkol M, Blau N, Andresen B S. 2012. Splicing of phenylalanine hydroxylase (PAH) exon 11 is vulnerable: molecular pathology of mutations in PAH exon 11. Mol Genet Metab. 106(4):403-11.
³ Sun C, Southard C, Di Rienzo A. 2009. Characterization of a novel splicing variant in the RAPTOR gene. Mutat Res. 9; 662(1-2):88-92.
⁴ Fukao T, Horikawa R, Naiki Y, Tanaka T, Takayanagi M, Yamaguchi S, Kondo N. 2010. A novel mutation (c.951C>T) in an exonic splicing enhancer results in exon 10 skipping in the human mitochondrial acetoacetyl-CoA thiolase gene. Mol Genet Metab. 100(4):339-44.
⁵ Gonçalves V, Theisen P, Antunes O, Medeira A, Ramos J S, Jordan P, Isidro G. 2009. A missense mutation in the APC tumor suppressor gene disrupts an ASF/SF2 splicing enhancer motif and causes pathogenic skipping of exon 14. Mutat Res. 662(1-2):33-6.
⁶ Burgess R, MacLaren R E, Davidson A E, Urquhart J E, Holder G E, Robson A G, Moore A T, Keefe R O, Black G C, Manson F D. 2009. ADVIRC is caused by distinct mutations in BEST1 that alter pre-mRNA splicing. J Med. Genet. 46(9):620-5.
⁷ Jensen C J, Stankovich J, Butzkueven H, Oldfield B J, Rubio J P. 2010. Common variation in the MOG gene influences transcript splicing in humans. J. Neuroimmunol. 229(1-2):225-31.
⁸ Tran V K, Takeshima Y, Zhang Z, Yagi M, Nishiyama A, Habara Y, Matsuo M. 2006. Splicing analysis disclosed a determinant single nucleotide for exon skipping caused by a novel intraexonic four-nucleotide deletion in the dystrophin gene. J Med Genet. 43(12):924-30.
⁹ Gabut M, Miné M, Marsac C, Brivet M, Tazi J, Soret J. 2005. The SR protein SC35 is responsible for aberrant splicing of the E1alpha pyruvate dehydrogenase mRNA in a case of mental retardation with lactic acidosis. Mol Cell Biol. 25(8):3286-94.
¹⁰ Colapietro P, Gervasini C, Natacci F, Rossi L, Riva P, Larizza L. 2003. NF1 exon 7 skipping and sequence alterations in exonic splice enhancers (ESEs) in a neurofibromatosis 1 patient. Hum Genet. 113(6):551-4.
¹¹ Raponi M, Kralovicova J, Copson E, Divina P, Eccles D, Johnson P, Baralle D, Vorechovsky I. 2011. Prediction of single-nucleotide substitutions that result in exon skipping: identification of a splicing silencer in BRCA1 exon 6. Hum Mutat. 32(4):436-44.

What is claimed is:

1. A method for assessing changes in expression level of a gene having an mRNA splice-altering mutation, said mutation being located within a sequence window circumscribing an exon and one or more intronic sequences of said gene, said one or more intronic sequences being adjacent to said exon, said method comprising the steps of:
(a) computing and identifying changes in individual information contents of potential donor and acceptor splice sites at each nucleotide position by computing product of the information theory-based position weight matrices and a unitary position matrix of each sequence,
(b) defining potential exons by selecting every pair combination of acceptor and donor splice sites in the sequence window, and determining the gap surprisal value based on distance in nucleotides between sites comprising a pair combination, wherein the gap surprisal value is calculated for each potential exon length based on frequency of said length in the genome as the inverse log₂ of said frequency,
(c) computing the total information content, R_{i,total}, of a potential exon as the sum of the corresponding individual information contents of the acceptor and donor pair, corrected by adding the gap surprisal of an exon whose length is the distance between the donor and acceptor pair,
(d) comparing the R_{i,total} values of all potential mRNA splice isoforms of the wild-type gene and the same values after the wild-type gene sequence is mutated to determine whether the mutation alters the abundance of the mRNA isoforms containing the exon, wherein the splice isoform with the largest R_{i,total} value is predicted to be the most abundant splice isoform, and the splice isoform with the smallest R_{i,total} value is the least abundant isoform, and
(e) extracting mRNAs or proteins from at least one cell expressing said gene to determine the most abundant mRNA splice isoform of said gene, thus allowing the assessing of changes in expression level of said gene.

2. The method of claim 1, wherein the comparison step (d) determines the relative abundance of a pair of splice isoforms by computing 2 to the power of the difference between the R_{i,total} values of each isoform.

3. The method of claim 2, wherein the mutation occurs at a cryptic splice site.

4. The method of claim 3, wherein the mutation is a leaky or partial splicing mutation, said mutation causing a mutant isoform to exceed the abundance of the normal mRNA splice isoform by at least 1 bit or 2 fold.

5. The method of claim 3, wherein a paucimorphic or effectively null allele for a splicing mutation occurs in which a mutant isoform exceeds the abundance of the normal mRNA splice isoform by at least 5 bit or 32 fold.

6. The method of claim 2, wherein the mutation occurs at a natural splice site.

7. The method of claim 6, wherein the mutation is a leaky or partial splicing mutation, said mutation causing the R_{i,total} of the mutant isoform to be less than the R_{i,total} value of the normal mRNA splice isoform by at least 1 bit or 2 fold.

8. The method of claim 6, wherein paucimorphic or effectively null allele for a splicing mutation occurs in which the R_{i,total} of the mutant isoform is less than the R_{i,total} value of the normal mRNA splice iso o m by at least 5 bits or 32 fold.

9. The method of claim 1, wherein the method is specific for first exons, using a first exon-specific gap surprisal function.

10. The method of claim 1, wherein the method is specific for last exons, using a last exon-specific gap surprisal function.

11. The method of claim 1, further comprising a step (f) of correcting the R_{i,total} from step (c) by taking into account one or more splicing enhancer and/or one or more silencer sequence elements recognized by an RNA binding protein or a small nuclear ribonucleoprotein, wherein strength of at least one of said splicing enhancer and/or said one or more silencer sequence elements is altered due to the mutation of said gene.

12. The method of claim 11, wherein a secondary gap surprisal is applied to take into account distances between the natural splice site and each of the altered splicing enhancer and/or silencer sequence elements.

13. The method of claim 12, wherein at least one weak binding site that overlaps with a stronger binding site is not taken into account when applying said secondary gap surprisal.

14. The method of claim 1, wherein effects on exon definition by said mutation at binding sites for an RNA binding protein are taken into consideration by correcting the total information content (R_{i,total}) by changes in strengths of the binding sites and by a gap surprisal, said gap surprisal being determined by scanning the genome for binding sites of said binding protein with a position weight matrices (PWM) to determine the frequency of each interval length between known natural sites and the nearest binding site for said RNA binding protein, separately for exons and introns, wherein said PWM is generated using known CLIP-seq libraries for said RNA binding protein.

15. The method of claim 1, wherein said step (e) is performed by extracting mRNAs from said at least one cell and by determining the sequence of one or more mRNA molecules derived from said gene.

16. The method of claim 1, wherein said step (e) is performed by extracting proteins from said at least one cell expressing said gene and by determining the sequence of one or more protein molecules derived from said gene.

17. A method for determining changes in expression level of a gene having an mRNA splice-altering mutation, said mutation being located within a sequence window circumscribing an exon and one or more intronic sequences of said gene, said one or more intronic sequences being adjacent to said exon, said method comprising the steps of:
(a) computing and identifying changes in the individual information contents of potential donor and acceptor splice sites at each nucleotide position by computing product of the information theory-based position weight matrices and a unitary position matrix of each sequence,
(b) defining potential exons by selecting every pair combination of acceptor and donor splice sites in the sequence window, and determining the gap surprisal value based on distance in nucleotides between sites comprising a pair combination, wherein, the gap surprisal value is calculated for each potential exon length based on frequency of said length in the genome as the inverse log₂ of said frequency,
(c) computing the total information content, R_{i,total}, of a potential exon as the sum of the corresponding individual information contents of the acceptor and donor pair, corrected by adding the gap surprisal of an exon whose length is the distance between the donor and acceptor pair,
(d) comparing the R_{i,total} values of all potential mRNA splice isoforms of the wild-type gene and the same values after the wild-type gene sequence is mutated to determine whether the mutation alters the abundance of the mRNA isoforms containing the exon, wherein the splice isoform with the largest R_{i,total} value is predicted to be the most abundant splice isoform, and the splice isoform with the smallest R_{i,total} value is the least abundant isoform, and
(e) introducing said gene into at least one cell and extracting mRNAs or proteins from said at least one cell expressing said gene to determine the most abundant mRNA splice isoform of said gene, thus allowing the assessing of changes in expression level of said gene.

18. The method of claim 17, further comprising a step (f) of correcting the R_{i,total} from step (c) by taking into account one or more splicing enhancer and/or one or more silencer sequence elements recognized by an RNA binding protein or a small nuclear ribonucleoprotein, wherein strength of at least one of said splicing enhancer and/or said one or more silencer sequence elements is altered due to the mutation of said gene.

19. The method of claim 18, wherein a secondary gap surprisal is applied to take into account distances between the natural splice site and each of the altered splicing enhancer and/or silencer sequence elements.

20. A method for determining changes in expression level of a gene having an mRNA splice-altering mutation, said mutation being located within a sequence window circumscribing an exon and one or more intronic sequences of said gene, said one or more intronic sequences being adjacent to said exon, said method comprising the steps of:
(a) generate a genomic polynucleotide sequence of the gene,
(b) computing and identifying changes in the individual information contents of potential donor and acceptor splice sites at each nucleotide position by computing product of the information theory-based position weight matrices and a unitary position matrix of each sequence,
(c) defining potential exons by selecting every pair combination of acceptor and donor splice sites in the sequence window, and determining the gap surprisal value based on distance in nucleotides between sites comprising a pair combination, wherein, the gap surprisal value is calculated for each potential exon length based on frequency of said length in the genome as the inverse log₂ of said frequency,
(d) computing the total information content, R_{i,total}, of a potential exon as the sum of the corresponding individual information contents of the acceptor and donor pair, corrected by adding the gap surprisal of an exon whose length is the distance between the donor and acceptor pair, and
(e) comparing the R_{i,total} values of all potential mRNA splice isoforms of the wild-type gene and the same values after the wild-type gene sequence is mutated to determine whether the mutation alters the abundance of the mRNA isoforms containing the exon, wherein the splice isoform with the largest R_{i,total} value is predicted to be the most abundant splice isoform, and the splice isoform with the smallest R_{i,total} value is the least abundant isoform, thus allowing the assessing of changes in expression level of said gene.

21. The method of claim 20, wherein the comparison step (e) determines the relative abundance of a pair of splice isoforms by computing 2 to the power of the difference between the R_{i,total} values of each isoform.

22. The method of claim 21, wherein the mutation occurs at a cryptic splice site.

23. The method of claim 22, wherein the mutation is a leaky or partial splicing mutation, said mutation causing a mutant isoform to exceed the abundance of the normal mRNA splice isoform by at least 1 bit or 2 fold.

24. The method of claim 22, wherein a paucimorphic or effectively null allele for a splicing mutation occurs in which a mutant isoform exceeds the abundance of the normal mRNA splice isoform by at least 5 bit or 32 fold.

25. The method of claim 21, wherein the mutation occurs at a natural splice site.

26. The method of claim 25, wherein the mutation is a leaky or partial splicing mutation, said mutation causing the R_{i,total} of the mutant isoform to be less than the R_{i,total} value of the normal mRNA splice isoform by at least 1 bit or 2 fold.

27. The method of claim 25, wherein paucimorphic or effectively null allele for a splicing mutation occurs in which the R_{i,total} of the mutant isoform is less than the R_{i,total} value of the normal mRNA splice isoform by at least 5 bits or 32 fold.

28. The method of claim 20, further comprising a step (f) of correcting the R_{i,total} from step (d) by taking into account one or more splicing enhancer and/or one or more silencer sequence elements recognized by an RNA binding protein or a small nuclear ribonucleoprotein, wherein strength of at least one of said splicing enhancer and/or said one or more silencer sequence elements is altered due to the mutation of said gene.

29. The method of claim 28, wherein a secondary gap surprisal is applied to take into account distances between the natural splice site and each of the altered splicing enhancer and/or silencer sequence elements.
